(19) United States
(12) Patent Application Publication
(10) Pub. No.: US 2018/0142300 A1
(43) Pub. Date: May 24, 2018

(54) UNIVERSAL HAPLOTYPE-BASED NONINVASIVE PRENATAL TESTING FOR SINGLE GENE DISEASES

(71) Applicant: The Chinese University of Hong Kong, Shatin (HK)

(72) Inventors: Wai In Hui, Diamond Hill (CN); Peiyong Jiang, Shatin (CN); Kwan Chee Chan, Shatin (CN); Yuk-Ming Dennis Lo, Homantin (CN); Rossa Wai Kwun Chiu, Shatin (CN)

(21) Appl. No.: 15/818,138

(22) Filed: Nov. 20, 2017

Related U.S. Application Data
(60) Provisional application No. 62/424,088, filed on Nov. 18, 2016.

(51) Int. Cl. C12Q 1/68

(52) U.S. Cl. CPC C12Q 1/6883; C12Q 2600/156; C12Q 2600/172

(57) ABSTRACT

To detect a fetal mutation inherited from the mother without paternal genetic information, a property of each maternal haplotype can be measured in the cell-free mixture. A separation value between values of the property for the two maternal haplotypes can be compared to thresholds to determine which haplotype is inherited. As measurements of a paternal allele may not be available, embodiments can measure the property at some loci where the fetus is homozygous and some loci where the fetus is heterozygous, but account for such loci where the fetus is heterozygous in the selection of a threshold for determining inheritance of a maternal haplotype. To determine parental haplotypes, direct haplotyping can be performed, and loci within a specified of the mutation can be selected and used in haplotype block for the measurements. Targeted measurements of a region including the mutation using predetermined primer/probes that may be re-used across subjects.

### CROSS-REFERENCES TO RELATED APPLICATIONS

The present application claims priority to and is a nonprovisional of U.S. Provisional Application No. 62/424,088, entitled “Universal Haplotype-Based Noninvasive Prenatal Testing For Single Gene Diseases” filed Nov. 18, 2016, the entire contents of which are herein incorporated by reference for all purposes.

### BACKGROUND

The presence of cell-free fetal DNA in maternal plasma (Lo Y M et al., Lancet 1997; 350:485-7) offers a noninvasive approach for prenatal diagnosis. Maternal plasma DNA analysis for the screening of common fetal chromosomal aneuploidies has been achieved with high degree of accuracy (Chiu R W et al. Bmj 2011; 342:c7401; McCullough R M et al., PLoS One 2014; 9:e109173) resulting in substantial reductions in the number of invasive prenatal diagnostic procedures performed.

Apart from fetal aneuploidies, single gene disease is the other reason why some pregnant women consider prenatal diagnosis. Since fetal DNA is present in a background of maternal DNA (Lun F M et al., Clin Chem 2008; 54:1664-72), early work for the noninvasive determination of single gene disease inheritance focused on the analysis of paternally transmitted fetal-specific sequences or mutations that could be distinguished from the maternal genome. For example, the detection of chromosome Y sequences in maternal plasma allowed accurate fetal sex determination and hence served as a means to evaluate the risk of a fetus for having a sex-linked disorder (Lo Y M et al., Am J Hum Genet 1998; 62:768-75; Costa J M, Benachi A, Gautier E, N Engl J Med 2002; 346:1502; Bustamante-Aragones A et al., Haemophilia 2008; 14:593-8). The presence or absence of paternally-inherited mutant alleles in maternal plasma has been applied to the noninvasive assessment of paternally inherited autosomal dominant diseases or for the exclusion of the fetus being affected by an autosomal recessive disease (Lo Y M et al. Prenatal diagnosis of fetal RhD status by molecular analysis of maternal plasma. N Engl J Med 1998; 339:1734-8; Saito H et al., Lancet 2000; 356:1170; Chiu R W et al., Lancet 2002; 360:998-1000).

However, the detection of certain paternally-inherited mutant alleles can be difficult, e.g., gene deletion, inversion, mutations in repetitive elements, and homologous genes, even with excessive depths of sequencing. Further, it can be difficult to detect maternally-inherited mutations, particularly if no genetic information is available from the father.

### SUMMARY

Embodiments can provide efficient and accurate techniques for measuring genomic properties of a fetus without invasively taking a sample directly from the fetus, which would otherwise carry a significant risk to the fetus. Instead, embodiments can analyze a cell-free mixture of fetal and maternal DNA fragments (e.g., plasma, serum, urine, and the like) obtained from the mother. The analysis can be performed in a particular manner to determine inheritance of a parental haplotype, which may include a mutation. Such techniques can be valuable to determine whether the fetus has inherited a mutation from a parent, where genetic treatment can be performed when the fetus has inherited the mutation.

Some embodiments can advantageously reduce the number of samples to be analyzed and/or a number of loci analyzed in the cell-free mixture. For example, the testing of samples from a father to obtain paternal genetic information can be avoided (e.g., to address situations where such information is not available), while still allowing a determination of an inheritance of a maternal haplotype from the mother in a given chromosomal region. In some implementations, to provide the technical ability to perform such a measurement without paternal genetic information, a property of each maternal haplotype can be measured in the cell-free mixture (e.g., counts or sizes of sequence reads having different alleles at loci in the chromosomal region). A separation value (e.g., a difference or ratio) between values of the property for the two maternal haplotypes can be compared to thresholds to determine which haplotype is inherited. As measurements of a paternal allele may not be available, embodiments can measure the property at some loci where the fetus is homozygous and some loci where the fetus is heterozygous, but account for such loci where the fetus is heterozygous in the selection of a threshold for determining inheritance of a maternal haplotype.

Some embodiments can advantageously reduce the number of samples to be analyzed by avoiding a need for a trio of samples (e.g., parents and a previous child) to perform haplotyping of the parents. To this end, DNA molecules that overlap with the chromosomal region and that are at least 1 kb long (or 5 kb, 10 kb, or 20 kb) can be sequenced in a cellular maternal sample to obtain long sequence reads from both chromosomal copies in the chromosomal region. Such long reads can be used to construct maternal and/or paternal haplotypes. To reduce a number of loci analyzed in the cell-free mixture, a mutation in a parent haplotype can be identified, and loci near the mutation and having certain characteristics (e.g., that parent is heterozygous) can be selected. For example, for inheritance of maternal haplotypes, the characteristic can include that the mother is heterozygous, but also that a paternal allele is known at a locus. As an example for inheritance of paternal haplotypes, in addition to the father being heterozygous at the selected loci, the characteristics can include that the mother is homozygous for first alleles of a first paternal haplotype at a first subset of the selected loci and that the mother is homozygous for second alleles of a second paternal haplotype at a second subset of the selected loci.

These and other embodiments of the invention are described in detail below. For example, other embodiments are directed to systems, devices, and computer readable media associated with methods described herein.

A better understanding of the nature and advantages of embodiments of the present invention may be gained with reference to the following detailed description and the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a high-level flowchart illustrating a method for indirectly detecting a mutation in a fetal genome that is inherited from a parent according to embodiments of the present invention.

FIG. 2 shows a schematic diagram of a technique 200 of haplotype phasing using linked-read sequencing according to embodiments of the present invention.

FIG. 3 is a flowchart illustrating a method 300 for detecting a mutation in a fetal genome of a fetus inherited from a father using a biological sample obtained from a pregnant mother of the fetus according to embodiments of the present invention.

FIG. 4 is a flowchart illustrating a method 400 for detecting a mutation in a fetal genome of a fetus inherited from a pregnant mother using a biological sample obtained from the pregnant mother according to embodiments of the present invention.

FIG. 5 shows a table 500 of the mutational statuses of the studied cases.

FIG. 6 shows a table of sequencing data of parental genomic DNA processed with the 10×™ system according to embodiments of the present invention.

FIG. 7 is a table 700 showing an overview of targeted sequencing data of maternal plasma DNA according to embodiments of the present invention.

FIG. 8 shows a table 800 of haplotype phasing data for families A-M according to embodiments of the present invention.

FIG. 9 shows fetal haplotype analyses in families A to F according to embodiments of the present invention.

FIG. 10A shows haplotype linkage to a mutation site (30 kb deletion) for the mother in family A. FIG. 10B shows haplotype linkage to a mutation site for the father in family A.

FIG. 11 is a table 1100 showing informative SNPs used for maternal plasma analysis according to embodiments of the present invention.

FIG. 12 shows the fetal haplotype analyses in families G to M according to embodiments of the present invention.

FIGS. 13A-13D illustrate haplotype assignment inferred by the presence of apparently long maternal DNA molecules. FIG. 13A shows normalized coverage of long molecules with reference to total depth across chrX. FIGS. 13B-13D shows boxplots of lengths of DNA molecules within (FIG. 13C) or outside (FIGS. 13B or 13D) the gene rearrangement regions.

FIG. 14 is a schematic illustration of a paternal-free size RHSO principle according to embodiments of the present invention.

FIGS. 15A-15C show representative size profiles between Hap I and Hap II according to embodiments of the present invention.

FIG. 16 shows a summary of PRHSO and PRHDO performance according to embodiments of the present invention.

FIG. 17 shows the correlation of the degree of imbalance between Hap I and Hap II reflected in size- and count-based analysis according to embodiments of the present invention.

FIGS. 18A and 18B show the factors affecting the minimal number of plasma DNA molecules required to achieve classification with a sensitivity of 95% according to embodiments of the present invention.

FIG. 19 shows the fold change in the number of plasma DNA molecules required for haplotype block classification when the fetal DNA fraction in the sample is doubled from 5%, 10%, 15%, or 20% according to embodiments of the present invention.

FIG. 20 is a table 2000 showing the theoretical number of molecules required in PRHSO and PRHDO analysis for the real cases according to embodiments of the present invention.

FIG. 21 shows a recombination identification with the use of sliding window based PRHDO according to embodiments of the present invention.

FIG. 22 shows results for PRHSO and PRHDO for an error-prone region according to embodiments of the present invention.

FIG. 23 is a flowchart of a method 2300 of determining a portion of a fetal genome of a fetus inherited from a pregnant mother using a biological sample obtained from the pregnant mother.

FIG. 24 illustrates a measurement system according to an embodiment of the present invention.

FIG. 25 shows a block diagram of an example computer system usable with system and methods according to embodiments of the present invention.

### TERMS

A “biological sample” may refer to any sample that is taken from a subject (e.g., a human, such as a pregnant woman, a person with cancer, or a person suspected of having cancer, an organ transplant recipient or a subject suspected of having a disease process involving an organ (e.g., the heart in myocardial infarction, or the brain in stroke, or the hematopoietic system in anemia) and contains one or more nucleic acid molecule(s) of interest. The biological sample can be a bodily fluid, such as blood, plasma, serum, urine, vaginal fluid, fluid from a hydrocele (e.g. of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g. thyroid, breast), etc. Stool samples can also be used. In various embodiments, the majority of DNA in a biological sample that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. The centrifugation protocol can include, for example, 3,000 g×10 minutes, obtaining the fluid part, and re-centrifuging at for example, 30,000 g for another 10 minutes to remove residual cells.

The term fractional fetal DNA concentration is used interchangeably with the terms fetal DNA proportion and fetal DNA fraction, and refers to the proportion of DNA molecules that are present in a maternal plasma or serum sample that is derived from the fetus (Lo Y M D et al. Am J Hum Genet 1998; 62:768-775; Lun F M F et al. Clin Chem 2008; 54:1664-1672).

A “sequence read” refers to a string of nucleotides sequenced from any part or all of a nucleic acid molecule. For example, a sequence read may be a short string of nucleotides (e.g., 20-150) sequenced from a nucleic acid fragment, a short string of nucleotides at one or both ends of a nucleic acid fragment, or the sequencing of the entire nucleic acid fragment that exists in the biological sample (a subtype of sequencing both ends). Sequencing both ends of the fragment can provide greater accuracy in the alignment and also provide a length of the fragment. A sequence read may be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

A “locus” or its plural form “loci” may refer to a location or address of any length of nucleotides (or base pairs) which has a variation across genomes.

The term “haplotype” as used herein refers to a combination of alleles at multiple loci that are transmitted together on the same chromosome or chromosomal region. A haplotype may refer to as few as one pair of loci or to a chromosomal region, or to an entire chromosome. The term “alleles” refers to alternative DNA sequences at the same physical genomic locus, which may or may not result in different phenotypic traits. In any particular diploid organism, with two copies of each chromosome (except the sex chromosomes in a male human subject), the genotype for each gene comprises the pair of alleles present at that locus, which are the same in homozygotes and different in heterozygotes. A population or species of organisms typically includes multiple alleles at each locus among various individuals. A genomic locus where more than one allele is found in the population is termed a polymorphic site. Allelic variation at a locus is measurable as the number of alleles (i.e., the degree of polymorphism) present, or the proportion of heterozygotes (i.e., the heterozygosity rate) in the population. As used herein, the term “polymorphism” refers to any inter-individual variation in the human genome, regardless of its frequency. Examples of such variations include, but are not limited to, single nucleotide polymorphism, simple tandem repeat polymorphisms, insertion-deletion polymorphisms, mutations (which may be disease causing) and copy number variations.

“Direct haplotyping” of a subject refers to haplotyping that does not require genetic information from another subject. Thus, the haplotyping can be performed using only a sample of the subject. In contrast, indirect haplotyping uses genetic information of another subject, such as a trio of parents and a child to determine a haplotype of a parent. Examples of direct haplotyping include single molecule sequencing, linked-read sequencing, and single molecule long-range PCR followed by detection of alleles by hybridization probes, microarray, mass-spectrometry and others.

The term “size profile” generally relates to the sizes of DNA fragments in a biological sample. A size profile may be a histogram that provides a distribution of an amount of DNA fragments at a variety of sizes. Various statistical parameters (also referred to as size parameters or just parameter) can be used to distinguish one size profile to another. One parameter is the percentage of DNA fragment of a particular size or range of sizes relative to all DNA fragments or relative to DNA fragments of another size or range.

The term “size distribution” refers to any one value or a set of values that represents a length, mass, weight, or other measure of the size of molecules corresponding to a particular group (e.g. fragments from a particular haplotype or from a particular chromosomal region). Various embodiments can use a variety of size distributions. In some embodiments, a size distribution relates to the rankings of the sizes (e.g., an average, median, or mean) of fragments of one chromosome relative to fragments of other chromosomes. In other embodiments, a size distribution can relate to a statistical value of the actual sizes of the fragments of a chromosome. In one implementation, a statistical value can include any average, mean, or median size of fragments of a chromosome. In another implementation, a statistical value can include a total length of fragments below a cutoff value, which may be divided by a total length of all fragments, or at least fragments below a larger cutoff value.

A “separation value” corresponds to a difference or a ratio involving two values. The separation value could be a simple difference or ratio. As examples, a direct ratio of x/y is a separation value, as well as x/(x+y). The separation value can include other factors, e.g., multiplicative factors. As other examples, a difference or ratio of functions of the values can be used, e.g., a difference or ratio of the natural logarithms (1n) of the two values. A separation value can include a difference and a ratio.

A “property” of a group of DNA fragments may refer to a quantitative and collective property, e.g., relating to a count or a size value of the group of DNA fragments. As examples, a value of the property can be the number of fragments in the group or a statistical value of a size distribution of the fragments in the group. The group of DNA fragments may belong to a same haplotype.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1). The terms “cutoff” and “threshold” refer to predetermined numbers used in an operation. For example, a cutoff size can refer to a size above which fragments are excluded. A threshold value may be a value above or below which a particular classification applies. Either of these terms can be used in either of these contexts.

### DETAILED DESCRIPTION

The discovery of cell-free fetal DNA (Lo, Y. M. D. et al. Lancet 350, 485-487 (1997)) and its miscellaneous applications in noninvasive prenatal testing (NIPT) have revolutionized prenatal care. The detection of fetal chromosomal aneuploidies (Chiu, R. W. K. et al. Proc Natl Acad Sci 105, 20458-20463 (2008); Fan, H. C. et al. Proc Natl Acad Sci 105, 16266-16271, (2008); Chiu R W et al. Bmj 2011; 342:c7401; Yu, S. C. et al. PLoS One 8, e60968 (2013); Strayer, R. et al. WISECONDOR Nucleic Acids Res 42, e31 (2014)), fetal microdeletions (Yu, S. C. Y. et al. Clinical chemistry, doi:10.1373/clinchem.2016.254813 (2016)), single gene diseases (Lam, K. W. et al. Clinical chemistry, doi: clinchem.2012.189589 [pii] 10.1373/clinchem.2012.189589 (2012); New M I et al., J Clin Endocrinol Metab 2014; 99:E1022-30) and fetal de novo mutations (Chan, K. C. et al. Proc Natl Acad Sci USA 113, E8159-E8168, (2016)) in a noninvasive manner can be achieved. In particular, NIPT for common chromosomal aneuploidies has been rapidly translated into clinical practice in more than 90 countries and was used by millions of pregnant women worldwide (Allyse, M. et al. Int J Womens Health 7, 113-126 (2015); Chandrasekharan, S. et. al., Sci Transl Med 6, 231fs215 (2014)).

Since whole-genome haplotyping technologies were not mature in the past, haplotype information was derived from analyzing samples of related family members such as a proband (New M I et al., J Clin Endocrinol Metab 2014; 99:E1022-30). However, this meant that for most practical purposes, the approach could only be applied to families where DNA from a previously affected member was available. With the use of direct haplotyping methods, such as linked-read sequencing, one can use the RHDO approach for noninvasive prenatal testing in families where no proband sample is available. Some embodiments have applied linked-read sequencing technology to directly generate haplotype-resolved genome sequence from parental DNA.

Maternal plasma DNA sequencing data were interpreted with the parental haplotype information to deduce the mutational status of the fetus by selecting particular loci using haplotype information from the parent and determining collective properties of sequence reads from the maternal plasma DNA at the selected loci. This protocol was used for the noninvasive prenatal assessment of a number of autosomal and X-linked diseases, showing that this streamlined approach enabled noninvasive detection of single gene disease inheritance without the need to design bespoke assays to assess mutations on a case-by-case basis (Lench N et al., Prenat Dia 2013; 33:555-62; Verhoef T I et al., Prenat Dia 2016; 36:636-42) and only required the use of specimens from the parents.

Further, some embodiments have been developed that do not require any paternal DNA information to determine maternal inheritance. Collective properties of both haplotypes can be determined from sequence reads obtained from plasma, and a separation value between the collective property values can be compared to different thresholds, respectively corresponding to inheritance of the two haplotypes. In this manner, the ability to detect inheritance of maternal haplotypes, as well as maternal mutations, can be more universally applicable due to the ease of constraints on the required measurements that are needed.

### I. Detection of Inherited Mutation in Fetal Genome

To assess the fetal inheritance of maternally transmitted mutations, approaches have been developed to compare the relative amounts of the mutant and wildtype alleles or haplotypes in maternal plasma. The relative mutation dosage approach directly measures the number of DNA molecules in maternal plasma that carry the mutant or wildtype alleles. For a mother who is a carrier of a mutation, equal amounts or skewed amounts between the two alleles in maternal plasma would provide an indication of whether the fetus is heterozygous or homozygous for either allele, respectively (Lun F M et al., Proc Natl Acad Sci 2008; 105:19920-5; Tsui N B et al., Blood 2011; 117:3684-91).

The relative haplotype dosage (RHDO) approach, on the other hand, allows the deduction of the fetal genotype by measuring the relative counts of single nucleotide polymorphism (SNP) alleles on haplotypes linked with the mutant allele and wildtype allele in maternal plasma DNA (Lo Y M et al., Sci Transl Med 2010; 2:61ra91). This method allows the indirect measurement of mutations that are more challenging to be detected by direct mutation-specific assays, such as gene deletion, inversion, mutations in repetitive elements and homologous genes (New M I et al., J Clin Endocrinol Metab 2014; 99:E1022-30). The RHDO method could be applied in a genome-wide (Lo Y M et al., Sci Transl Med 2010; 2:61ra91) or a targeted fashion specifying the analysis for particular loci (New M I et al., J Clin Endocrinol Metab 2014; 99:E1022-30; Lam K W et al., Clin Chem 2012; 58:1467-75).

In RHDO analysis, maternal haplotype information is required. However, haplotype phasing strategies used in previous studies were complicated and laborious. Methods to determine haplotype information include inferential statistical analysis and direct experimental techniques. By genotyping genomic DNA of trios, including the father, mother and an affected proband in the family, SNPs linked with mutation sites could be identified and thus haplotypes could be deduced (New M I et al., J Clin Endocrinol Metab 2014; 99:E1022-30). This approach restricts the application of the testing to families with a previously affected family member whose DNA is available. Alternatively, haplotypes could be constructed by population-based inference (Zeevi D A et al., J Clin Invest 2015; 125:3757-65) or reconstructed from genomic DNA of an individual by methods such as clone pool dilution sequencing (Kitzman J O et al., Nat Biotechnol 2011; 29:59-63), contiguity-preserving transposition sequencing (Amini S et al., Nat Genet 2014; 46:1343-9) and HaploSeq (Selvaraj S, J R D, Bansal V, Ren B., Nat Biotechnol 2013; 31:1111-8). However, these techniques require intricate experimental protocols or reagents that are not yet widely commercially available (Snyder M W, Adey A, Kitzman J O, Shendure J., Nat Rev Genet 2015; 16:344-58).

A. Overview Using Direct Haplotyping for Detection of Inherited Mutation

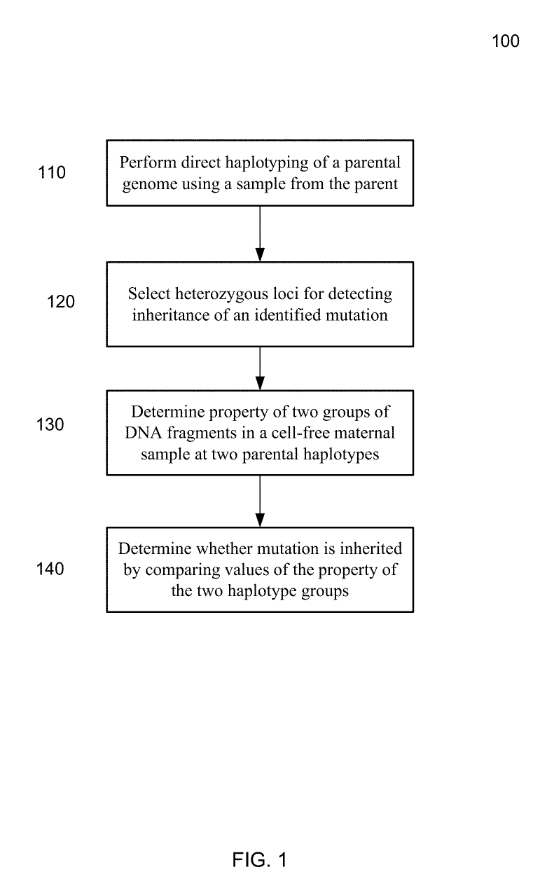

FIG. 1 is a high-level flowchart illustrating a method 100 for indirectly detecting a mutation in a fetal genome that is inherited from a parent according to embodiments of the present invention. The mutation can be from the mother or the father. Method 100 can use a sample from a parent for haplotyping, and then perform sequencing of a cell-free sample from the mother.

At block 110, direct haplotyping of a parental genome is performed using a sample from the parent. For example, the direct haplotyping can include sequencing DNA from a cellular sample, such as the white blood cells in a buffy coat of a blood sample. The direct haplotyping allows a reduction in the number of samples to be analyzed, since genetic information from a child (i.e., other than the fetus whose genome is not known) is not required. Examples of direct haplotyping include single molecule sequencing and linked-read sequencing.

As part of the direct haplotyping, long DNA molecules (e.g., 1 kb, 5 kb, 10 kb, 20 kb, 50 kb, 100 kb, or more) can be sequenced. Such long DNA molecules can result from a fragmentation process of cellular DNA, where the fragmentation process provides a significant portion of DNA molecules that are over 1 kb long. Long sequence reads corresponding to the long DNA molecules can be aligned to a reference genome to identify reads that overlap with a same chromosomal region. Long reads that have the same alleles at heterozygous loci can be used to reconstruct the haplotypes.

In some embodiments, a direct haplotype phasing approach uses microfluidics-based linked-read sequencing technology became available (Zheng G X et al., Nat Biotechnol 2016; 34:303-11). For example, long input DNA molecules can be partitioned into droplets and transformed into short barcoded fragments for sequencing. Identical barcodes are used to identify short fragments that originate from the same droplet, where such short fragments (reads) that are located near each other (e.g., in a reference genome) can be identified as being from a same long DNA molecule. In some implementations, a group of short fragments can be considered near each other when each short read in the group overlaps with at least one other short read. In other implementations, the short reads may just need to be within a specified distance of another short read, e.g., within 10, 50, 100, 200, 500, 1,000, 2,000, 3,000, 4,000, 5,000, 6,000, 7,000, 8,000, 9,000, 10,000, 20,000, 30,000, 40,000, 50,000, 60,000. 70,000, 80,000, 90,000 or 100,000, 200,000, 300,000, 400,000, 500,000, 600,000, 700,000, 800,000, 900,000, 1,000,000 bases.

When the amount of DNA in a sample is relatively diluted (e.g., spread across more droplets than there are genomic equivalents in the sample), it is unlikely that two long fragments are present from both haplotypes. Thus, an assumption of nearby short reads being from a same long DNA molecule can be made. Accordingly, reconstruction of the short-reads can provide long-range haplotype information.

At block 120, a set of heterozygous loci for detecting inheritance of an identified mutation is selected. The parent is heterozygous at these loci so that it can be determined which haplotype is inherited by analyzing reads from a cell-free maternal sample. Loci near the identified mutation can be selected since the mutation is likely inherited on a same haplotype. In various embodiments, loci can be selected that are within 100 bp, 1 kb, 10 kb, 100 kb, 1 Mb, or 5 Mb of the mutation.

The mutation can be identified in a particular chromosomal region of the parent. The direct haplotyping can be performed genome-wide or for a specific chromosomal region. When performed genome-wide, haplotypes in a particular chromosomal region can be selected. As described in more detail below, the selection of the set of loci can be performed in stages, e.g., selecting SNPs for a targeted analysis around a known disease, and then using data from a certain subset of those SNPs that have specific characteristics. In this manner, the same protocols and reagents across patients for detecting the inheritance of a same mutation.

In some embodiments, further criteria can be used to select the set of loci. For example, when genetic information of the other parent is known, loci where the other parent is homozygous can be selected. In this manner, the allele inherited from the other parent can be known. Further, the other parent can be homozygous for all the alleles of a same haplotype, e.g., first alleles of a first haplotype at the set of loci. In other embodiments, such genetic information of the other parent is not available, and thus is not used. In such situations, a selection of a threshold for determining inheritance can be modified, as is described in a later section.

At block 130, values of a property of the two groups of DNA fragments in a cell-free maternal sample at the two parental haplotypes are determined. The cell-fee maternal sample includes fetal and maternal DNA fragments, and the properties can reflect the inherited haplotype. For example, maternal plasma DNA can be subjected to sequencing, and SNP alleles located upstream and downstream of a disease locus can be identified. The haplotype origin of each SNP allele can be deduced. The sequence may be targeted, as may be done with capture probes or primers specific to the set of heterozygous loci. Such targeted sequencing can be done in combination with alignment to only the heterozygous loci, thereby providing more efficient sequencing and computational alignment.

The properties can be determined by identifying reads having the different alleles corresponding to the two parental haplotypes at the set of heterozygous loci. For example, sequence reads that align to the set of heterozygous loci in a reference genome can be identified and separated into two groups: a first group having one of first alleles corresponding to a first parental haplotype and a second group having one of second alleles corresponding to a second parental haplotype. For efficiency, the alignment of the sequence reads can be performed to only the set of heterozygous loci, and sequence reads not aligning to one of the heterozygous loci can be discarded.

One example of a property of a group of DNA fragments corresponding to a parent haplotype include the number of molecules in the group. A value of the property can be a normalized value, e.g., a count of the DNA fragments aligning to a haplotype divided the total number of DNA fragments for the sample or the number of DNA fragments for a reference region (e.g., a chromosome).

Another example of a property of a group of DNA fragments corresponding to a parent haplotype include a statistical value of a size distribution of the DNA fragments in the group. Example statistical values include an average, mean, or median size of DNA fragments in the group, as well as a total length or number of DNA fragments in one size range (e.g., below a size cutoff value or at a particular size, such as 150 bp), which may be divided by a total length or number of DNA fragments in a second range (e.g., all DNA fragments or DNA fragments below a larger size cutoff value).

At block 140, whether the mutation was inherited is determined by comparing values of the property of the two haplotype groups. If the haplotype with the identified mutation is inherited, then the mutation can be determined to be inherited. For example, a separation value can be determined between the two values for the two groups. In some embodiments, a difference or ratio between the two numbers of DNA fragments in the two groups can be determined, as may be done for relative haplotype dosage (RHDO). If the difference (e.g., HapI−HapII) exceeds a threshold, then the first parental haplotype can be identified as being inherited. The specific threshold and classification of inheritance can depend on whether information from the other parent is used, e.g., whether the other parent is homozygous for which set of alleles at the set of loci. Accordingly, a statistical comparison between the abundance of plasma DNA molecules derived from the two parental haplotypes can be performed to determine the inheritance.

In other embodiments, a separation value (e.g., a difference or ratio) between the two statistical values of a size distribution of DNA fragments in the two groups can be determined, as may be done for relative haplotype-based size shortening analysis (RHSO). Further details are provided herein.

When haplotyping is performed for both parents, an inherited haplotype for both the mother and father can be determined. The fetal genotype can then deduced based on the two sets of statistical results.

B. Direct Haplotyping

In some embodiments, parental haplotypes can be determined using microfluidics-based linked-read sequencing (Zheng G X et al., Nat Biotechnol 2016; 34:303-11) on blood cell DNA obtained from the pregnant woman and her male partner. Other sources of genomic DNA from either parent, such as DNA from buccal smear, buccal swabbing, hair follicular cells, etc., can be used. The linked-read sequencing of the parental DNA could be performed in a whole genome manner or could target specific disease-relevant loci. Methods of direct haplotyping other than linked-read sequencing, such as single molecule sequencing of long DNA molecules, can also be used. Alternatively, long-range PCR (Arbeithuber B et al, Methods Mol Biol 2017; 1551:3-22) of a single molecule of long DNA fragments and followed by means, for example by hybridization probes, microarray, mass spectrometry, to determine the alleles present on the DNA molecule would also produce direct haplotypes.

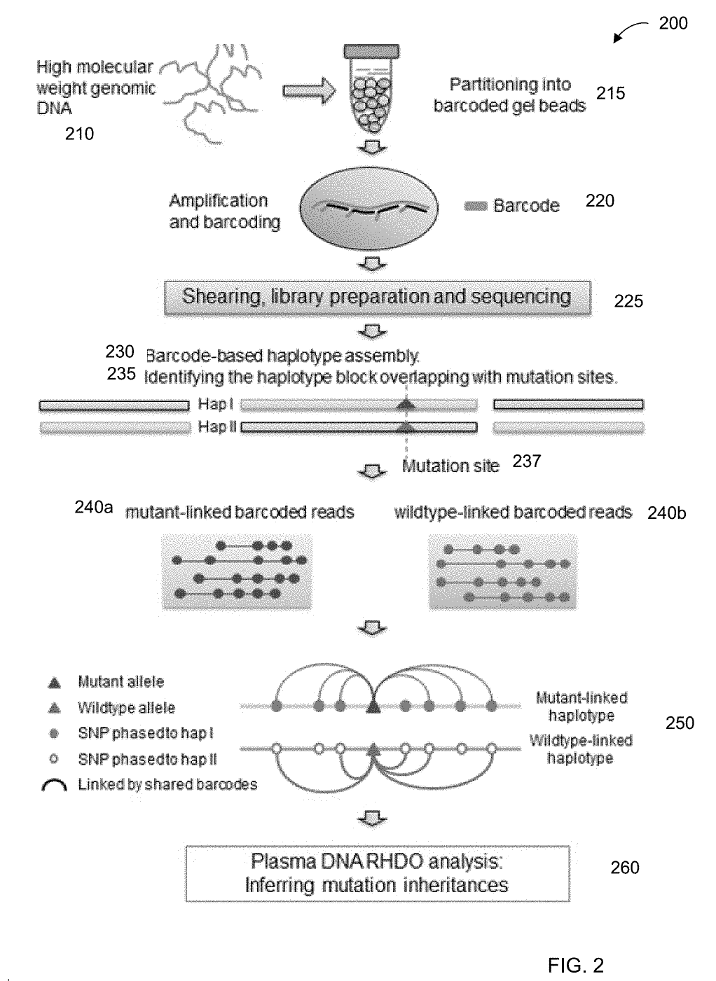

FIG. 2 shows a schematic diagram of a technique 200 of haplotype phasing using linked-read sequencing according to embodiments of the present invention. Technique 200 can be performed for either parent. As an example, from the parental buffy coat sequencing data, barcode information of each sequence read was used to link short sequence reads into original long input molecules. With sufficient dilution, the chance of having two distinct long DNA molecules that cover a genomic locus with opposing haplotype in the same partition, e.g., in a same well, same gel bead, or any other reaction vessel is very low.

Long DNA molecules 210 can be obtained from a tissue sample, e.g., a buffy coat of a parent. In various embodiments, intact cellular DNA in a nucleus can be fragmented via sonication or just by pipetting to obtain long DNA molecules 210. Depending on the process to obtain such DNA fragments, some long fragments and some shorter DNA fragments may be produced. In such situations, long DNA molecules 210 can be selected, e.g., by various filtration techniques, such as electrophoresis. In various implementations, fragments of 1 kb, 5 kb, 10 kb, or 20 kb and more are selected.

At 215, long DNA molecules 210 were partitioned into gel beads. A certain number of genomic equivalents of high molecular weight (HMW) genomic DNA can be distributed across many more droplet partitions. Given the number of beads and the number of long DNA molecules 210, the number of long DNA molecules in each bead would be sufficiently low so that no more than one long DNA molecule from any one genomic region would be represented in the same bead. Each bead could contain more than one long DNA molecules but none of the long DNA molecules in the same bead are from the same genomic locus. For example, each bead can have 1% of a genomic equivalent.

The gel beads can include barcoded oligonucleotides. Oligonucleotides having the particular barcode of a given gel bead can be attached to the DNA in that bead, for later identification purposes.

At 220, long DNA molecules 210 are fragmented, and the shorter DNA fragments are tagged with the barcoded oligonucleotides in a bead. The DNA fragmentation and barcode addition could be performed as one step, such as by tagmentation (Zhang et al, Nat Biotechnol 2017; 35:852-857). In some implementations, the fragmentation can be performed by subjecting the DNA to random priming and polymerase amplification. Such amplification will result in forward and reverse priming at random locations, and thus the amplicons will be of various sizes, e.g., several hundred to several kilobases. The resulting amplicons can be barcoded or the random primers contain the barcodes. In some implementations, long DNA molecules 210 can be amplified by 10×™ barcoded primers. This can be done by a process called multiple displacement amplification (MDA) or other amplification technologies with the use of random primers having barcode sequences.

At 225, barcode-tagged short DNA molecules are sequenced. The sequencing may be performed via various techniques, such as flowing over a sequencing cell and performing bridge amplification using adapters ligated to the ends of the barcode-tagged short DNA molecules. The sequencing could be performed by semiconductor sequencing, single molecule sequencing or any techniques that could determine the base sequence of a short piece of DNA. A detection system can detect signals (e.g., imaging of fluorescent signals or capturing of electrical signal) corresponding to different bases, thereby obtaining sequence reads. A sequence read can include the sequence of the short DNA molecules and the sequence of a barcoded oligonucleotide.

In some embodiments, after a random primer-mediated barcoding process, the DNA molecules may still be relatively long. In such situations, shearing DNA may be performed. But, shearing can be omitted, e.g., if multiple displacement amplification generated enough short fragments with barcode information.

At 230, short sequence reads that share the same barcode (e.g., from a same gel bead) are identified. The short sequence reads having a same barcode can be compared to each other, e.g., by aligning to a reference sequence, which may be an entire reference genome or a region that is being targeted. If a set of short sequence reads with the same barcode are near each other (e.g., overlap or are within a specified distance), then this set of reads can be identified as belonging to a same long DNA molecule. A set of nearby reads can be combined to reconstruct the sequence of the long DNA molecule for a given region. There may be multiple long reads in a given gel bead. Reconstructed long reads (across the gel beads) that overlap with each other (e.g., as determined by alignment to a reference) and have identical sequences in the overlapped region can be joined together as an extended haplotype. Accordingly, haplotype phasing of genomic DNA was achieved by initially linking short read sequencing data and subsequently joining overlapping assembled stretches of long DNA to provide long range genetic information.

At 235, the haplotype block overlapping with a mutation site 237 is identified. The haplotype block can correspond to a chromosomal region (e.g., as may be defined by a set of heterozygous loci). If multiple mutation sites are present in the parental genome, then multiple haplotype blocks can be identified.

As an example, a mutant allele at a particular location can be identified in the short sequence reads (e.g., after aligning to a reference). As part of the haplotype phasing, a set of short sequence reads sharing a same barcode that was present on reads carrying the mutant allele can be linked (mutant-linked barcode reads) and phased to the same haplotype (termed Hap I or mutant-linked haplotype). The reads having the mutant allele can be required to be in the set of nearby reads, and thus assumed to be part of the same long DNA molecule.

Similarly, wildtype-linked barcode reads were phased to the opposite haplotype. Accordingly, reads that shared the same barcode with the ones carrying wildtype alleles can be phased to the opposite haplotype (termed Hap II or wildtype-linked haplotype).

At 240a, a group of mutant-linked barcoded reads is shown. Each of these long sequence reads are from a different gel bead, and the circles can correspond to an allele at a heterozygous locus. Collectively, these alleles can be considered as first alleles of a first parental haplotype—Hap I or mutant-linked haplotype in this case.

At 240b, a group of wildtype-linked barcoded reads is shown. Each of these long sequence reads are from a different gel bead, and the circles can correspond to an allele at a heterozygous locus. Collectively, these alleles can be considered as second alleles of a second parental haplotype—Hap II or wildtype-linked haplotype in this case.

At 250, SNPs linked on the same haplotypes with the mutant and wildtype alleles were identified as a set of loci, e.g., as part of block 120 of method 100. The set of loci can be used in subsequent maternal plasma DNA analysis (e.g., RHDO or RHSO). In various embodiments, the SNPs can be within 100 bp, 1 kb, 10 kb, 100 kb, 1 Mb, or 5 Mb of the mutation. The window of the set of loci around the mutation can be asymmetric, e.g., if the mutation is near the end of a haplotype block, there may be more loci to the left of the mutation and farther away on the left.

At 260, sequencing is performed on the cell-free sample and reads are quantified (e.g., count or size). For example, SNP information on the mutant- or wildtype-linked haplotype can be extracted for RHDO or RHSO analysis.

In other embodiments, the direct haplotyping can use a recombination event (e.g., a large deletion, insertion, or inversion of 1 kb or more) on a chromosome copy to determine reads that are from a same haplotype. For example, the paired ends of the sequenced maternal DNA molecules that contained the recombinant would appear to be as long as HMW DNA molecules when mapped to the reference genome. However, in actuality, a fragmentation process can ensure that the fragments are on average smaller than 1 kb. On the basis of lengths determined from alignment, DNA fragments determined to be longer than a specified length (e.g., 1 kb, 5 kb, 10 kb, 20 kb, or more) can be considered to be from the haplotype having the recombination event. Accordingly, this feature can be used to assign SNPs to the respective haplotypes, namely SNP alleles associated with the apparently long DNA molecules were assigned to the mutant-linked haplotype.

C. Selecting Loci to Detect Mutation and Use of Probes/Primers

As described above, the selection of the set of loci can occur in multiple stages. For example, an initial set of loci can correspond to SNPs that are known to be near a certain disease locus, e.g., based on public database or sequencing of other subjects. The direct haplotyping in block 110 can use targeted sequencing that uses sequence-specific probes and/or sequence-specific primers to sequence the initial set of loci. Then, after the haplotypes are determined and the mutation is positively identified in a parental haplotype, certain loci where the parent is actually heterozygous (i.e., the parent may not be heterozygous at all of the loci of the initial set) can be selected. Further, the analysis of the cell-free maternal sample in block 130 can be performed using targeted sequencing with the probes and/or primers for the initial set, but only reads at the final selected loci can be used. In this manner, the target capture can be performed using the same protocols and reagents across patients.

Accordingly, an advantage of haplotype-based methods over direct mutational analysis is that one could infer the fetal inheritance through quantitative assessment of informative SNP alleles in maternal plasma, obviating the need for tailor-made mutation-specific assays (Lench N et al., PrenatDiagn 2013; 33:555-62; Verhoef T I et al., PrenatDiagn 2016; 36:636-42). Such tailor-made assays need to be optimized in good time to meet the requirements for a clinically acceptable turnaround time during pregnancy. Sometimes, mutation-specific assays cannot be as readily developed for some challenging genomic loci (e.g. repetitive regions, existence of homologous genes) or for certain mutations (deletions, inversions, gene recombinants). CYP21A2 is one such example, for which results are provided below. The sequences of CYP21A2 share high homology with the pseudogene CYP21A1P. Because the fetal genotype was inferred from the SNP allelic ratios in maternal plasma, assays tailor-made for the CYP21A2 mutations were not needed.

A series of probes for the target capture of SNPs surrounding of a group of clinically important single gene disease loci could be pre-stocked in the laboratory. The scale of the testing could be varied depending on clinical needs. For example, one may elect to use only target capture probes designed for the assessment of one disease locus at a time. This strategy is suitable for the assessment of high risk pregnancies either with a family history for a specific single gene disease or had been identified to be mutation carriers through screening programs (Samavat A, Modell B., Bmj 2004; 329:1134-7). Alternatively, target capture probes relevant for several disease loci could be pooled and be analyzed concurrently. This alternative strategy is useful when there are a number of gene loci to be tested, such as for the purpose of investigating fetal abnormalities, like congenital cardiac defects, detected by ultrasonography.

There is also the potential to apply this noninvasive testing approach in the public health setting aimed at the prenatal management of diseases that are of high prevalence in the community, for example cystic fibrosis, sickle cell anemia or the thalassemias, or diseases that would benefit from prenatal (New M I, Abraham M, Yuen T, Lekarev O., Semin Reprod Med 2012; 30:396-9) or early neonatal treatment. When used as a public health screening tool, the capture probes can first be used for carrier identification (Bell C J et al., Sci Transl Med 2011; 3:65ra4) where the linked-read sequencing of the parental DNA is used to determine the parental mutations and haplotype structures. The same probes can then be used for the target capture of maternal plasma DNA for haplotype-based fetal genotype assessment. Thus, the workflow for the prenatal screening and detection of single gene diseases can be streamlined.

Various criteria can be used to further select loci for detecting a mutation, e.g., at a second selection stage. The proximity of the loci to the mutation is one criterion. Another example criterion is the parent being heterozygous at the loci, e.g., as determined based on the direct haplotyping. A further criterion can be that the inherited allele from the other parent can be deduced, e.g., (1) based on the other parent being homozygous at the set of loci or (2) based on paternal-specific alleles being detected at certain loci and an inherited paternal haplotype being selected from a plurality of reference haplotypes. Additionally, the number of loci in the set can be required to be at least a specified number.

For determining inheritance of a paternal haplotype, informative loci (e.g., SNPs) where the mother was homozygous and the father was heterozygous can be analyzed. Each of such informative loci would be specific to a particular paternal haplotype, namely the one having the unique allele. For example, if the mother is homozygous for A/A and the father is heterozygous for A/G (with paternal Hap II having G), then such an informative locus would be informative for Hap II. Such informative loci can be identified by genotyping the mother, but also by analyzing the allelic content of the cell-free mixture at those loci. Embodiments can assume the mother is homozygous when the allelic fraction of one allele is less than a specific percentage (e.g., 25%, 20%, 15%, or 10%).

The loci where there is such a paternal-specific alleles can be tracked and roughly an equal percentage of informative loci specific to each of the two haplotypes can be selected for testing. If the fetus had inherited the mutation from father, reads with the paternal-specific alleles detected in the cell-free maternal sample (e.g., plasma or serum) would belong to the paternal mutant-linked haplotype as identified by the haplotype analysis of the paternal DNA. In particular, the number of reads having a paternal allele from one of a first set of N informative loci specific to the mutant-linked haplotype can be compared to the number of reads having a paternal allele from one of a second set of N informative loci specific to the wild-type haplotype.

For determining inheritance of a maternal haplotype, informative loci (e.g., SNPs) where the mother was heterozygous and father was homozygous can be analyzed. Each SNP can be classified as type α or type β. These two types can be considered two different set of loci, with each set being using independently. In other implementations, each type of loci can be considered different subset of a same set of loci, e.g., where the different group of DNA fragments correspond to different subsets of loci.

For type α SNPs, the paternal alleles are identical to the maternal alleles on the maternal mutant-linked haplotype. If the fetus had inherited the mutant allele, an overrepresentation of mutant-linked haplotype would be observed in maternal plasma DNA. In contrast, if the fetus had inherited the wildtype allele, there would be no overrepresentation of either one of the maternal haplotypes. For type β SNPs, the paternal alleles are identical to the maternal alleles on the maternal wildtype-linked haplotype, i.e. haplotype linked with the wildtype allele. If the fetus had inherited the wildtype haplotype, an overrepresentation of wildtype-linked haplotype would be observed. On the other hand, if the fetus had inherited the mutant allele, both haplotypes would be equally represented.

D. Properties of Haplotypes

Various properties of the sequence reads from the cell-free mixture can be used to distinguish a presence of a particular haplotype. Depending on which haplotype is inherited from the parent being analyzed, the properties of the two haplotypes in the cell-free mixture will be different, thereby indicating which haplotype is inherited. Example properties include amounts of number of DNA molecules from each of the parental haplotypes (e.g., as determining from alignment) and a statistical value of a size distribution.

1. Amount of DNA Fragments at each Haplotype

Noninvasive prenatal testing for single gene disorders can be achieved by measuring a dosage imbalance of the DNA molecules that carried SNP information in maternal plasma. A principle of RHDO analysis is to assess the number of plasma DNA fragments that contain the SNP information linked to the mutant- and wildtype-associated haplotypes in the mother, respectively. The maternal haplotype transmitted to the fetus is expected to be over-represented relative to the other maternal haplotype. An amount of DNA fragments from each of the haplotypes at the selected set of loci can be counted based on which allele the DNA fragment has. An amount can be determined for each locus or a collective count for the set of loci can be used. Then, a separation value can be determined using the amounts, with the separation value indicating which haplotype is inherited.

In various embodiments, the amounts can be a number of fragments with a particular allele at one of the set of loci, a number of fragments from any of the set of loci on a particular haplotype, and a statistical value of a count (e.g., an average) at loci on a particular haplotype. Instead of number, a total length of the DNA fragments could also be used. Further examples can be found in U.S. Patent Publications 2011/0105353 and 2013/0040824, which are incorporated by reference in their entirety.

When a total count is determined for each haplotype, the individual counts at each locus of a haplotype are effectively aggregated before making a comparison. The aggregated amounts of the parental haplotypes can then compared to determine if a haplotype is over-represented, equally represented, or under-represented. In other implementations, the two amounts for fragments with the two alleles at a locus are compared, where comparisons at multiple loci can be used to aggregate individual separation values to obtain an aggregate separation value.

When a count is determined for each locus, a running sum can be determined for each haplotype, and a test can be determined using the sum after each locus to determine whether the separation value has sufficient statistical power to identify which haplotype is inherited. In some implementations, for maternal inheritance, two separation values can be determined, e.g., when type α and type β SNPs are used. Each separation value can be used to determine a separate classification of which haplotype is inherited. The two classifications can be compared to confirm consistency.

As described herein, a difference is one example of a separation value. For instance, separation value can be N_{hapI}-N_{hapII}, where N_{hapI} is the number of reads corresponding to the first haplotype, and N_{hapII} is the number of reads corresponding to the second haplotype. As another example, a ration of N_{hapI} and N_{hapII} can be used.

2. Size

The data analyses of NIPT were mainly based on counting the DNA molecules in maternal plasma (Lun F M et al., Proc Natl Acad Sci 2008; Lo Y M et al., Sci Transl Med 2010; Tsui N B et al., Blood 2011). Recently, it was demonstrated that the plasma DNA size properties can also be applied to detect fetal chromosomal aneuploidies (Yu, S. C. et al. Proc Natl Acad Sci of USA, 111, 8583-8588, doi:10.1073/pnas.1406103111 (2014)). The size-based approach takes advantage of the biological characteristics that the fetally-derived DNA molecules are shorter than the maternally-derived ones (Lo Y M et al., Sci Transl Med; Yu, S. C. et al. Proc Natl Acad Sci of USA, 111; Chan, K. C. et al. Clin Chem 50, 88-92, doi:10.1373/clinchem.2003.024893 (2004)) in maternal plasma. The presence of an extra fetal chromosome in fetal trisomy would result in additionally more short DNA fragments derived from the affected chromosome. In a later study (Yu, S. C. Y. et al. Clinical chemistry, doi:10.1373/clinchem.2016.254813 (2016)), it has been reported that the size-based analysis could also be used as an independent method to confirm the sub-chromosomal copy number aberrations (CNAs) detected by count-based analysis. The combined analysis of size- and count-based analyses could reduce the false positives and differentiate whether the aberrations are maternal or fetal derived. A recent study demonstrated the possibility to utilize the size characteristics of the cell-free fetal DNA in maternal plasma to confirm the count-based analysis results and to differentiate whether the aberrations are of fetal or maternal origin, as described in U.S. Patent Publication 2016/0217251, which is incorporated by reference in their entirety.

A feasibility in conducting size-based analysis to deduce the fetal inheritance of maternally transmitted single gene disorders is explored herein. Specifically, embodiments explore the feasibility of a size-based approach, called the Relative Haplotype-based Size shOrtening analysis (RHSO), to deduce the fetal inheritance of maternal transmitted single gene mutations.

Because of the size difference between the fetally- and maternally-derived DNA molecules in maternal plasma, we reasoned that the presence of the fetally-derived maternally transmitted haplotype would alter the size distributions of the plasma DNA molecules originated from the two maternal haplotypes respectively. Therefore, we proposed that it might be possible to determine the maternal inheritance of the fetus by comparing statistical values of size distribution (e.g., the cumulative frequencies) of the two haplotypes at a particular size with the use of RHSO.

Various statistical values can be used to measure a relative difference in a size distribution of two haplotypes as a result of the shorter fetal DNA fragments in the cell-free mixture corresponding to the haplotype inherited by the fetus. Examples are provided herein, as well as in U.S. Patent Publications 2011/0276277 and 2013/0237431, which are incorporated by reference in their entirety. In some embodiments, RHSO analysis compares the cumulative frequencies of the DNA molecules that carried the single nucleotide polymorphisms on the two maternal haplotypes at a particular size (e.g., 150 bp). The cumulative frequency can be measured as a total percentage of DNA fragments at a size or smaller out of all of the DNA fragments measured.

3. Targeted Analysis

In some embodiments, a targeted analysis of the cell-free mixture can be performed to obtain a sufficient number of reads for accurately determining the values of the property of the two haplotypes, thereby ensuring adequate statistical accuracy. In some circumstances, noninvasive deduction of the fetal genotype can be achieved when the maternal plasma DNA data surrounding the disease locus are adequate to allow statistically significant dosage assessments between the parental haplotypes. The amount of sequence information needed is dependent on the fetal DNA fraction, the number of loci in the selected set of loci (e.g., informative SNPs), and the sequencing depth.

If a sufficient number of reads are not obtained, additional capture probes and/or primers targeting a particular disease locus could be redesigned to capture more SNPs. Computational simulation showed that if the number of SNPs reached 1000 with 200-fold sequencing depth, statistically confident RHDO classifications can be generated even with low fractional fetal DNA concentrations (New M I et al., J Clin Endocrinol Metab 2014; 99:E1022-30).

E. Determination of Inheritance from Difference in Properties

Sufficiently different values for a property (e.g., count of DNA fragments or statistical size value) for the two parental haplotypes provide an accurate indication of which haplotype is inherited. The separation value between the two property values for the two groups of DNA fragments (i.e., for the two haplotypes) can be compared to a threshold to determine whether the indication is sufficiently strong. For example, a threshold can be used to confirm the over-representation of one haplotype.

1. Paternally Transmitted Autosomal Mutations

An amount of reads corresponding to each haplotype can be computed. For each haplotype, reads having a paternal-specific allele (i.e., not found in the mother) can be counted to determine an amount. Different subsets of the selected loci can be used, depending on which haplotype the paternal-specific allele is on (i.e., the mother is homozygous for the allele on the other haplotype). For example, a first subset of loci can have first alleles from a first paternal haplotype, and a second subset of loci can have second alleles from a second paternal haplotype. The existence of such loci can be determined by genotyping the mother or analyzing the relative allelic fractions of alleles at various loci, e.g., as described above.

For noninvasive prenatal testing (NIPT) applications developed in the past, the fetal inheritance of any paternal-specific alleles could simply be based on the presence or absence of that allele in maternal plasma. In embodiments of the present invention, a statistical test (e.g., the Kolmogorov-Smirnov test (KS test)) is used to statistically compare the accumulated allelic counts between the two subsets of paternal alleles. With the use of a statistical comparison between the paternal haplotypes, embodiments can minimize the chance of inadvertently making a misjudgement of the fetal inheritance due to sequencing errors. For example, sequencing error may result in a base change that happens to correspond to the allele on the paternal haplotype that the fetus did not inherit. Allelic counts of informative SNPs along one of the paternal haplotypes can be cumulatively counted sequentially until the counts along a region of one haplotype is statistically significantly elevated compared with counts from the corresponding region of the other paternal haplotype. In this manner, the chance of some erroneous bases resulting from sequencing artefacts resulting in an incorrect judgement of the fetal haplotype could be minimized. Another advantage of performing statistic comparison between the paternal haplotypes is that locations of recombination events that may occur between paternal haplotypes could be pinpointed with higher precision.

Accordingly, the KS test can be applied to determine whether there is a statistical difference of allelic counts between the two paternal haplotypes. Read counts of paternal-specific alleles between paternal haplotypes can be respectively accumulated until a mutant-linked haplotype or a wildtype-linked haplotype was classified (New M I et al., J Clin Endocrinol Metab 2014; 99:E1022-30). To minimize stochastic influences, the haplotype block can be required to fit certain criteria, e.g., the number of SNPs in the test chromosomal region ≥25; the cumulative difference between two haplotypes >0.53%; and the p-value of the KS test <0.05 (New M I et al., J Clin Endocrinol Metab 2014; 99:E1022-30). As to cumulative difference, it is the number of reads with paternal-specific alleles linked to paternal Hap I and Hap II that are different from maternal homozygous alleles. If the fetus inherited the paternal Hap I, then there should be M reads having the paternal Hap I specific alleles (e.g., from a first subset of loci) and N reads having the paternal Hap II specific alleles (e.g., from a second subset of loci), where M>N. Because there may be some sequence errors that are identical alleles on paternal Hap I or Hap II, embodiments can set a minimal cumulative difference between paternal Hap I and Hap II specific alleles to overcome the influence caused by sequencing errors. The percentage difference can be determined as M-N divided by a total number of reads (i.e., including maternal alleles) at the two subsets of loci.

2. RHDO Analysis of Maternally Transmitted Autosomal Mutations

In some embodiments, a RHDO analysis based on sequential probability ratio test (SPRT) classification can be performed to deduce the fetal inheritance of the maternally transmitted mutations (Lo Y M et al., Sci Transl Med 2010; 2:61ra91; New M I et al., J Clin Endocrinol Metab 2014; 99:E1022-30). The RHDO analysis can involve a statistical evaluation of dosage balance or imbalance between alleles to determine the haplotype block inherited.

The RHDO analysis can be performed using select loci, e.g., type α SNPs or type β SNPs. The separation value for each type corresponds to different determinations of which haplotype is inherited. For example, for type α SNPs, an over-representation of reads from the mutant haplotype (e.g., separation value is greater than a threshold) indicates that the mutant haplotype is inherited, while a roughly equal representation of reads between the two haplotypes (e.g., separation value is below a threshold) indicates that the wild-type haplotype is inherited. For type β SNPs, an over-representation of reads from the wild-type haplotype (e.g., separation value is greater than a threshold or below a negative threshold) indicates that the wild-type haplotype is inherited, while a roughly equal representation of reads between the two haplotypes (e.g., separation value is below a threshold) indicates that the mutant haplotype is inherited.

Various statistical tests can be used to determine the suitable thresholds, e.g., the sequential probability ratio test (SPRT) can be used. In some embodiments, the null hypothesis for each SPRT classification was that the dosage of the two maternal haplotypes was balanced. For type α SNPs, the alternative hypothesis was the overrepresentation of mutant-linked haplotype. For type β SNPs, the alternative hypothesis was the underrepresentation of mutant-linked haplotype. An odds ratio of 1200 (fold change between the chance of Hap I transmitted to the fetus versus Hap II transmitted to fetus) may be used to calculate the threshold for accepting or rejecting the null hypothesis. The equations calculating the thresholds were described previously (Lo Y M et al., Sci Transl Med 2010; 2:61ra91).

The RHDO block classification can start from the mutation site and extended towards the neighboring upstream and downstream SNPs. The upstream and downstream can be done as separation classifications, or loci (e.g., SNPs) can be selected alternately from each direction. Read counts of SNPs along a RHDO block can be accumulated until a mutant-linked haplotype or a wildtype-linked haplotype was classified. To minimize biases caused by hybridization and mapping efficiency, SNPs that had skewed read counts beyond 95% confidence interval between opposite haplotypes were filtered out (i.e., not used) because such a difference between two alleles is far deviated from the expected deviation caused by fetus's contribution, which is more likely caused by extra analytical biases such as hybridization and/or mapping efficiency. The 95% confidence interval can be deduced according to Poisson or binomial distribution by fitting the current sequencing depth of each SNP site. The unexpected skewness of read counts between two alleles can be also defined by using 99%, 90%, 85%, 80%, 75%, 70%, 65%, 60% confidence intervals.

As described in the later section on paternal-free techniques, some embodiments may not determine a type of a locus, thereby not requiring genetic information about the father.

3. RHSO

In some embodiments, similar types of loci can be used as for RHDO, e.g., type α SNPs or type β SNPs. The statistical size values for RHSO can measure a relative proportion of short DNA fragments to large DNA fragments, as specified by different size ranges, which may be 1 bp wide. When a maternal haplotype is inherited, the proportion of small DNA fragments will increase, and thus there can be a relationship between dosage representation and a statistical size value.

In RHDO using type α SNPs, the over-representation of reads from the mutant haplotype indicates that the mutant haplotype is inherited. For RHSO, a higher proportion of small fragments for the mutant haplotype than the wild-type haplotype (e.g., separation value between size values is greater than a threshold) indicates that the mutant haplotype is inherited, whereas roughly equal size values between the two haplotypes (e.g., separation value is below a threshold) indicates that the wild-type haplotype is inherited. For type β SNPs, a higher proportion of small fragments for the wild-type haplotype than the mutant haplotype (e.g., separation value is greater than a threshold or below a negative threshold) indicates that the wild-type haplotype is inherited, while roughly equal size values between the two haplotypes (e.g., separation value is below a threshold) indicates that the mutant haplotype is inherited.

Examples size values include the fraction of total length contributed by short DNA fragments can be calculated as follows:

F=Σ^{w}length/Σ⁶⁰⁰length, where
Σ^{w} length represents sum of the lengths of DNA fragments with length equal to or less than cutoff w (bp) for a given haplotype; and
Σ⁶⁰⁰ length represents the sum of lengths of the DNA fragments equal to or less than 600 bp for a corresponding group of the haplotype. Large cutoff values other than 600 bp can be used. A criteria can be that the two ranges are different, although they may overlap. The separation value ΔF can be F(Hap I)−F(Hap II), where Hap I or Hap II can be defined as the mutant haplotype. Other examples are F(Hap II)−F(Hap I), F(Hap I)/F(Hap II).

Another example size value is a fraction of short DNA fragments is used. One sets a cutoff size (w) to define the short DNA molecules. The cutoff size can be varied and be chosen to fit different diagnostic purposes. A computer system can determine the number of DNA fragments from a haplotype that are equal to or shorter than the size cutoff. The fraction of DNA fragments (Q) can then be calculated by dividing the number of short DNA by the total number of DNA fragments for that haplotype. The value of Q would be affected by the size distribution of the population of DNA molecules. A shorter overall size distribution signifies that a higher proportion of the DNA molecules would be short fragments, thus, giving a higher value of Q. Q_{HapI} and Q_{HapII} are examples of a statistical value of the two groups of the size distributions of fragments from each of the haplotypes. Examples separation values are similar as above, e.g., ΔQ=Q_{HapI}−Q_{HapII}, ΔQ=Q_{HapII}−Q_{HapI}. ΔQ=Q_{HapI}/Q_{HapII}, or ΔQ=Q_{HapII}/Q_{HapI}.

Another example of cumulative frequency at a given size is also described herein. Additionally, techniques using RHSO can also be used when genetic information about the father is not known.

4. Statistical Analysis for the Assessment of X-Linked Inheritance

The statistical analyses for the detection of inherited mutations on an autosome vs. on chromosome X can differ. For example, informative SNPs on chromosome X where mother was heterozygous can be analyzed. If a male fetus had inherited the mutation, there would be an overrepresentation of reads aligning to the mutant-linked haplotype from the cell-free mixture (e.g., a maternal plasma DNA analysis). If a male fetus had inherited the wild-type allele, there would be an underrepresentation of reads aligning to the mutant-linked haplotype (i.e., an over-representation of reads aligning to the wildtype-linked haplotype).

The two alternative hypotheses can be tested: (a) the mutant allele was overrepresented when compared to the wild-type allele, and (b) the mutant allele was underrepresented when compared to the wild-type allele (Tsui N B et al., Blood 2011; 117:3684-91). Various statistical tests can be used, e.g., SPRT, binomial test, Poisson test, Chi-square test, and Fisher exact test.

5. Measurement of Fractional Fetal DNA Concentration

In some embodiments, a fractional fetal DNA concentration can be used to determine threshold values, as the fractional concentration of fetal DNA can affect the extent of separation between the values for the two haplotypes. However, such usage is not required. For cases where both the paternal and maternal genomic DNA samples were sequenced, the fractional fetal DNA concentration in maternal plasma (f) can be calculated based on SNPs that were homozygous in both parents but for different alleles (Lo Y M et al., Sci Transl Med 2010; 2:61ra91).

$$f = \frac{\sum 2 p}{\sum ( p + q )} ,$$

where p is the read count of the fetal-specific allele and q is the read count of the allele shared by the maternal and fetal genomes.

For families at risk for an X-linked disease, the fractional fetal DNA concentration can be determined as follows. The homologous ZFY and ZFX gene loci located on chromosomes Y and X can be quantified, respectively, with the use of droplet digital PCR (ddPCR) technology. The primer and probe composition were described previously (Tsui N B et al., Blood 2011; 117:3684-91). The reaction for one sample (2 panels) was set up with the ddPCR Supermix for Probes (Bio-Rad) in a reaction volume of 20 μL according to the manufacturer's protocol and mixed with 70 μL droplet generation oil (Bio-Rad) using a QX100 or QX200 Droplet Generator (Bio-Rad). The reactions were initiated at 37° C. for 30 min for the action of uracil N-glycosylase, followed by 95° C. incubation for 10 min, 50 cycles of 94° C. for 30 s and 57° C. for 1 min, and 1 cycle of 98° C. for 10 min. Droplets were then loaded into the QX200 Droplet Reader (Bio-Rad). The concentration of ZFY and ZFX were calculated by QuantaSoft Software version 1.7.4 (Bio-Rad). The Fractional fetal DNA concentration=(2×ZFY)/(ZFY+ZFX)×100%, where ZFY and ZFX are the concentration of the ZFY and ZFX molecules.

F. Method for Detecting Mutations

As described above, embodiments can detect whether a mutation on a particular haplotype is inherited by the fetus, without having to take a direct sample from the fetus (e.g., via amniocentesis or chorionic villus sampling). Instead, a maternal sample comprising a cell-free mixture of fetal and maternal DNA is used, thereby allowing the measurement of whether the mutation is inherited.

1. Father

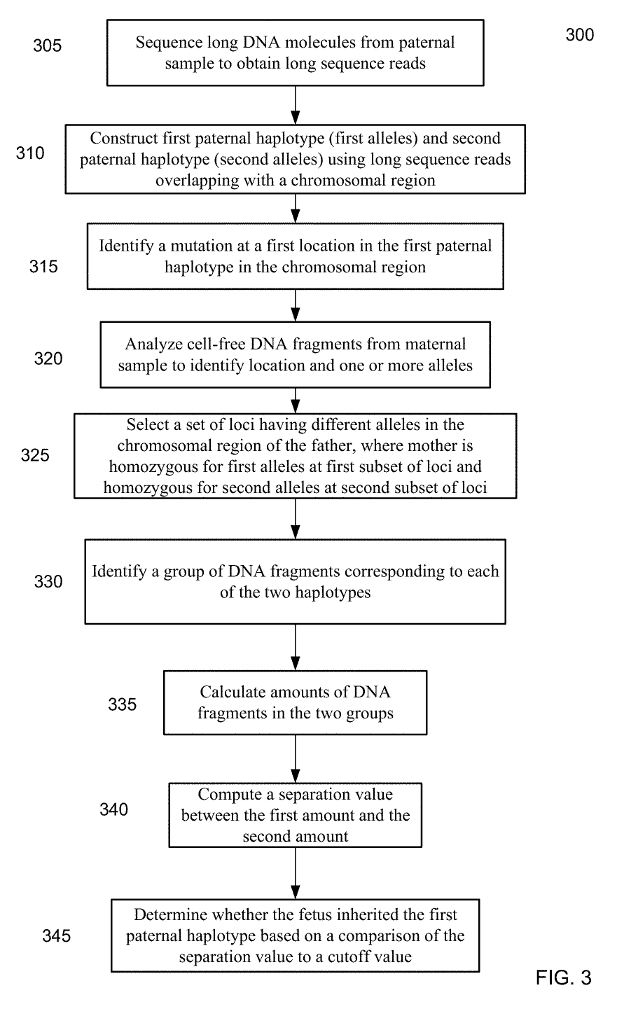

FIG. 3 is a flowchart illustrating a method 300 for detecting a mutation in a fetal genome of a fetus inherited from a father using a biological sample obtained from a pregnant mother of the fetus according to embodiments of the present invention. The mutation may be a cause of a single-gene disorder. The father has a paternal genome with a first paternal haplotype and a second paternal haplotype in a chromosomal region, which can be identified before or after applying an assay to a paternal sample. The biological sample contains a mixture of maternal and fetal DNA fragments, thereby allowing an non-invasive measurement but making such measurement more difficult than using a fetal sample. A mutation may or may not already be identified in a paternal genome prior to direct haplotyping of a paternal sample.

At block 305, long DNA molecules in a cellular paternal sample (e.g., a buffy coat of a blood sample) are sequenced to obtain long sequence reads. The sequencing can specifically target DNA molecules in a particular chromosomal region (e.g., which includes a mutation that is being measured as part of the assay). In one implementation, the sequencing can be genome-wide, but only long DNA molecules that overlap with the particular chromosomal region can be selected for further analysis. The long sequence reads would be from both chromosomal copies in the chromosomal region that is being haplotyped. For the long DNA molecules and the corresponding long sequence reads to be considered long, a requirement can be at least 1 kb, 5 kb, 10 kb, 20 kb, 50 kb, or 100 kb in length.

At block 310, the first and second paternal haplotypes are constructed using the long sequence reads that overlap with the chromosomal region, which has a mutation. Long sequence reads that overlap with the chromosomal region can be identified by alignment to a reference. The first paternal haplotype can be constructed using a first set of long sequence reads that share alleles at a plurality of loci in the chromosomal region, where the first paternal haplotype has first alleles at the plurality of loci. The second paternal haplotype can be constructed using a second set of long sequence reads that share alleles at the plurality of loci in the chromosomal region, where the second paternal haplotype has first alleles at the plurality of loci.

The reconstruction of a haplotype can identify long reads that overlap at one or more loci that are heterozygous in the father. These heterozygous loci can be identified from the allelic counts at various loci (e.g., were allelic percentage is greater than 40% for each of the two alleles at the locus). Long reads that have the same alleles at heterozygous loci (i.e., the long reads overlap and have a same sequence in the overlapping region) can be used to reconstruct the haplotypes. The number of loci in the overlap region where two long reads have the same alleles can be required to be at least a specified number (e.g., 2, 5, 10, etc.), such that sufficient amount of matching is confirmed in the overlap region. In this manner, having the same alleles at these heterozygous loci indicates that those long reads are on a same haplotype, and thus can be used to determine overlap regions with other long reads, thereby extending the haplotype.

As another example, population haplotypes can be employed to extend the parental haplotypes. For instance, one population haplotype block showing a high LD (linkage disequilibrium) value (e.g. >0.95) and sharing the same alleles with parental haplotype blocks deduced from direct haplotyping approaches can allow the parental haplotype blocks to be linked together to form longer haplotype blocks.

At block 315, a mutation is identified at a first location in the first paternal haplotype in the chromosomal region. The mutation may already be known to be at the first location, which can be one of the heterozygous loci used to reconstruct the haplotypes. Once the haplotypes are known, a particular haplotype with the mutation can be identified as a mutant haplotype.

At block 320, a plurality of cell-free DNA fragments are analyzed from the biological sample obtained from the pregnant mother. The maternal sample contains a mixture of maternal and fetal nucleic acids. The maternal sample can be taken, potentially refined, (e.g., purified for cell-free DNA), and then received for analysis, e.g., subjected to an assay and analyzing the resulting sequence data. In various embodiments, the maternal sample can be plasma, serum, urine, saliva, or uterine lavage fluid.

In some embodiments, analyzing a DNA fragment can include identifying a location of the DNA fragment in a reference genome (e.g., a reference human genome when the subject is human—other animals can be tested). An allele of the DNA fragment can be determined, e.g., when the DNA fragment overlaps a heterozygous locus. The analyzing can be performed in various ways, such as DNA sequencing, microarrays, hybridization probes, fluorescence-based techniques, optical techniques, molecular barcodes and single molecule imaging (Geiss G K et al. Nat Biotechnol 2008; 26: 317-325), single molecule analysis, PCR, digital PCR, mass spectrometry, etc. Any method that will allow the determination of the genomic location and allele (information as to genotype) of DNA fragments in the maternal biological sample can be used. Some of such methods are described in U.S. Patent Publication 2010/0112590, which is incorporated by reference in its entirety.

The analysis may specifically target a genomic window that includes the mutation. For example, primers can amplify DNA in the genomic window, and then sequencing can be performed. As another example, probes can preferentially capture DNA within the genomic window. In various implementations, such captured DNA can be sequenced or signals specific to a probe can indicate an allele of the capture DNA fragment at one of the set of selected loci.

At block 325, a set of loci are selected from a plurality of loci, e.g., heterozygous loci used to determine the haplotypes. The set of loci can be selected based on the first location of the mutation and based on a maternal genome of the pregnant mother being homozygous at the set of loci. The set of loci can be selected within a specified distance of the first location of the mutation. A proximity distance can be various values, e.g., as provided herein.

Two different types of loci can be determined based on which allele the mother is homozygous, i.e., type γ loci and type ζ loci. The maternal genome can be homozygous for the first alleles at a first subset (type γ) of the set of loci, and the maternal genome can be homozygous for the second alleles at a second subset (type ζ of the set of loci. Accordingly, probes and/or primers that are specific to a genomic window that includes the mutation can be used.

At block 330, groups of DNA fragments corresponding to each of the haplotypes are identified. For example, a first group of DNA fragments in the biological sample can be identified as having one of the first alleles at one of the first subset of loci based on the identified locations and the determined alleles for the first group of DNA fragments. The first group can include at least one DNA fragment located at each of the first subset of loci. A second group of DNA fragments in the biological sample can be identified as having one of the second alleles at one of the second subset of loci based on the identified locations and the determined alleles for the second group of DNA fragments. The second group can include at least one DNA fragment located at each of the second subset of loci.

At block 335, an amount of DNA fragments in teach of the two groups is are calculated. For example, a computer system can calculate a first amount of the first group of DNA fragments, and the computer system can calculate a second amount of the second group of DNA fragments. Such amounts are example values of a property of a haplotype, as is described herein. As examples, the amounts can be numbers of DNA fragments or total length of the DNA fragments of a group.

At block 340, a separation value between the first amount and the second amount is computed. Examples of separation values are provided herein, e.g., including a difference or a ratio. The separation value can allow a determination of which of the two haplotypes is represented more than the other.

At block 345, it is determined whether the fetus inherited the mutation on the first paternal haplotype based on a comparison of the separation value to a cutoff value. It can further be determined whether the fetus inherited the second paternal haplotype. The determination can be made using various statistical tests, e.g., the Kolmogorov-Smirnov test, Fisher's exact test, Poisson test, and binomial test.

2. Mother

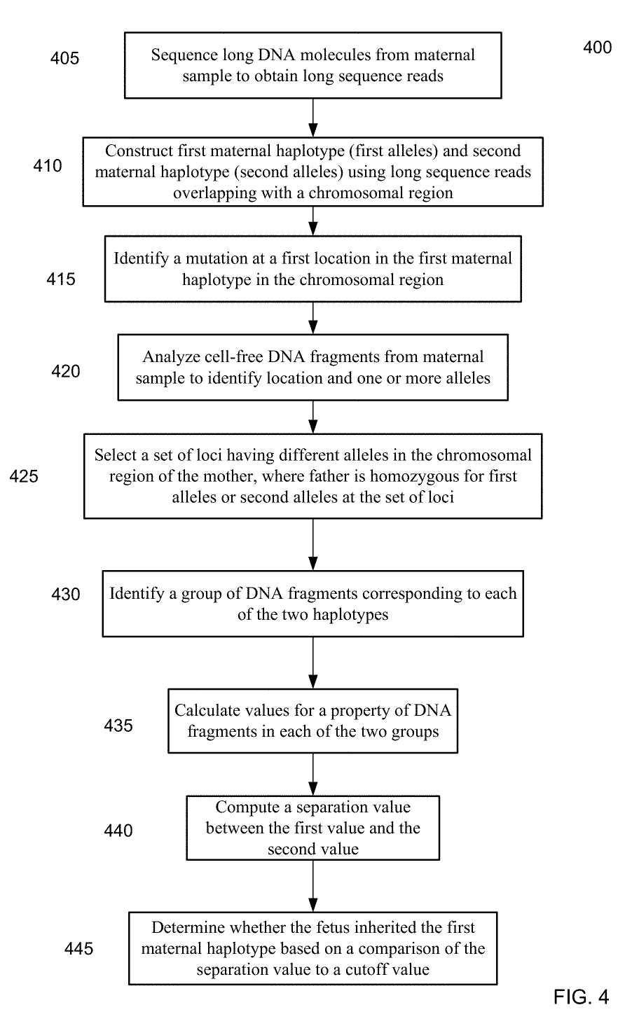

FIG. 4 is a flowchart illustrating a method 400 for detecting a mutation in a fetal genome of a fetus inherited from a pregnant mother using a biological sample obtained from the pregnant mother according to embodiments of the present invention. The mutation may be a cause of a single-gene disorder. The pregnant mother has a maternal genome with a first maternal haplotype and a second maternal haplotype in a chromosomal region, which can be identified before or after applying an assay to a maternal sample.

Aspects of method 400 can be performed in a similar manner as in method 300. For example, the biological sample contains a mixture of maternal and fetal DNA fragments, thereby allowing an non-invasive measurement of the fetal mutational status. A mutation may or may not already be identified in the maternal genome prior to direct haplotyping of a maternal sample.

At block 405, long DNA molecules in a cellular maternal sample (e.g., a buffy coat of a blood sample) are sequenced to obtain long sequence reads. Block 405 may be performed in a similar manner as block 305 of FIG. 3.

At block 410, the first and second maternal haplotype are constructed using the long sequence reads that overlap with the chromosomal region, which has a mutation. Block 410 may be performed in a similar manner as block 310 of FIG. 3. For example, the first maternal haplotype can be constructed using a first set of long sequence reads that share alleles at a plurality of loci in the chromosomal region, where the first maternal haplotype has first alleles at the plurality of loci. The second maternal haplotype can be constructed using a second set of long sequence reads that share alleles at the plurality of loci in the chromosomal region, where the second maternal haplotype has second alleles at the plurality of loci.

At block 415, a mutation is identified at a first location in the first maternal haplotype in the chromosomal region. Block 415 may be performed in a similar manner as block 315 of FIG. 3.

At block 420, a plurality of cell-free DNA fragments are analyzed from the biological sample obtained from the pregnant mother. Block 420 may be performed in a similar manner as block 320 of FIG. 3.

At block 425, a set of loci are selected from a plurality of loci (e.g., heterozygous loci used to determine the haplotypes) based on the first location of the mutation. Block 425 may be performed in a similar manner as block 325 of FIG. 3. Block 425 may further include determining paternal alleles inherited by the fetus from a father at the set of loci. The paternal alleles can correspond to the first alleles or the second alleles, e.g., corresponding to type α loci or type β loci. The set of loci can be selected based on locations that the paternal alleles are determined. Thus, the inherited paternal allele can be determined first, and then the set of loci selected. In various embodiments, a subset of type α loci or type β loci can be selected, and each one used separately.

The deduction of the inherited allele from the father can be deduced in various ways. For example, the inherited allele can be deduced based on the other parent being homozygous at the set of loci. As another example, the inherited allele can be deduced based on paternal-specific alleles being detected at certain loci and an inherited paternal haplotype being selected from a plurality of reference haplotypes.

At block 430, groups of DNA fragments corresponding to each of the haplotypes are identified. Block 430 may be performed in a similar manner as block 330 of FIG. 3. For example, a first group of DNA fragments can be identified as corresponding to the first maternal haplotype based on each of these DNA fragments having one of the first alleles. A second group of DNA fragments can be identified as corresponding to the second maternal haplotype based on each of these DNA fragments having one of the second alleles.

At block 435, a property of DNA fragments in each of the two groups is calculated. Examples of such a property are described herein, such as an amount of DNA fragments or a statistical value of a size distribution. Values of the property can be computed. For example, a computer system can calculate a first value of the first group of DNA fragments, where the first value defines a property of the DNA fragments of the first group. The computer system can also calculate a second value of the second group of DNA fragments, where the second value defines a property of the DNA fragments of the second group. In various embodiments, the properties can be determined according to RHDO or RHSO.

In some embodiments, the values can also be normalized values, e.g., a read count of the chromosomal region divided the total number of reads for the sample or the number of reads for a reference region. The values can also be a difference or ratio from another value (e.g., in RHDO), thereby providing the property of a difference for the region.

At block 440, a separation value is computed between the first value and the second value. Block 440 may be performed in a similar manner as block 3340 of FIG. 3.

At block 445, it is determined whether the fetus inherited the mutation on the first maternal haplotype based on a comparison of the separation value to a cutoff value and based on whether the paternal alleles correspond to the first alleles or the second alleles. It can further be determined whether the fetus inherited the second paternal haplotype. The determination can be made using various statistical tests, e.g., SPRT.

As an example, the determination can be based on the paternal alleles in that type α loci and type β loci can be treated differently. For example, a positive separation value above a first cutoff value for type α loci can indicate the first maternal haplotype is inherited, and thus the fetus inherited the mutation. A separation value near 0 for a difference or near 1 for a ratio (i.e., of the two values) can indicate that the second maternal haplotype is inherited. For type β loci, a negative separation value below a second cutoff value can indicate inheritance of the second maternal haplotype, while a separation value near 0 for a difference or near 1 for a ratio can indicate the first maternal haplotype is inherited, and thus the fetus inherited the mutation.

G. Results for Detecting Mutations

Various results using direct haplotyping of parental samples and mutation detection via an inherited haplotype are provided. The examples in this section using only RHDO and not RHSO; however, a later section for paternal-free techniques to determine an inherited maternal haplotype.

Thirteen families at risk for a fetus with congenital adrenal hyperplasia (CAH), beta-thalassemia, Ellis-van Creveld syndrome (EVC), hemophilia, or Hunter syndrome were recruited. Except for the pregnancy affected by EVC, each of the recruited families had a known family history of the disease for which conventional prenatal diagnosis was sought. For the EVC case, ultrasound examination revealed multiple structural abnormalities that led to the suspicion of EVC. The disease status of the fetus was determined by conventional prenatal assessment based on mutational analysis of the parental DNA and the fetal DNA, which was obtained by chorionic villus sampling or amniocentesis or after delivery by cord blood or newborn DNA analysis.

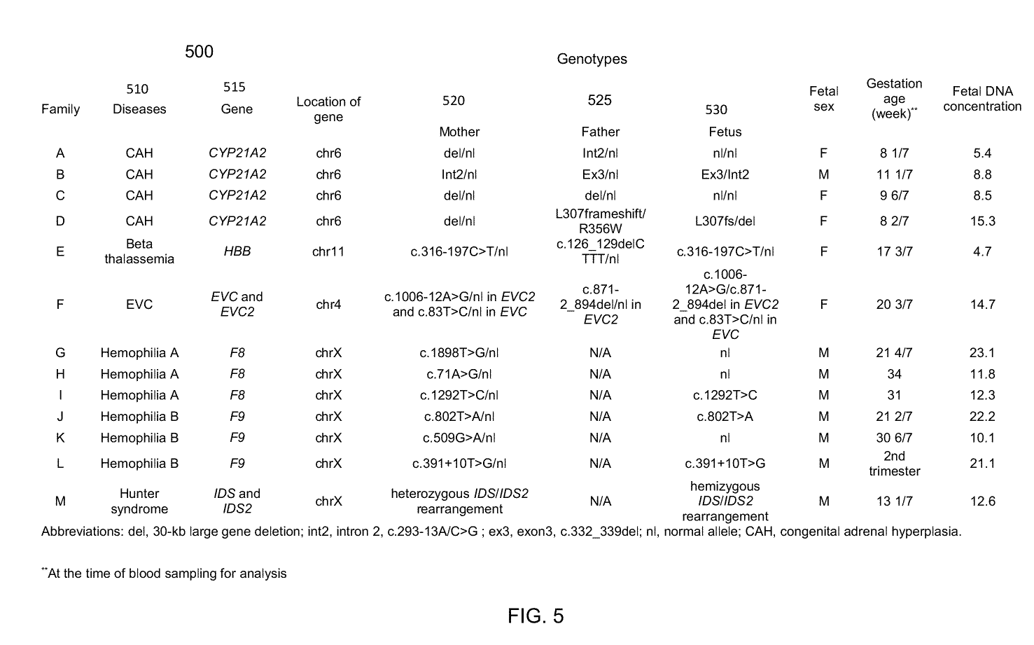

FIG. 5 shows a table 500 of the mutational statuses of the studied cases. The thirteen families are listed as A-M. The diseases are listed in column 510, and the gene corresponding to the disease is provided in column 515. Columns 520, 525, and 530 respectively show the genotypes of the mother, the father, and the fetus. In these column, the abbreviations are as follows: del is a 30-kb large gene deletion; int2 is for c.293-13A/C>G at intron 2; ex3 is for c.332_339del at exon3; and n1 is for normal allele. The gestation age is at the time of blood sampling for analysis.

For the CAH families, linked short reads were prepared from the parental buffy coat DNA that were target captured and sequenced to an average of 646-fold haploid human coverage. The capture probes target the major histocompatibility complex class III that contains the 21-hydroxylase (CYP21A2) gene (New M I et al., J Clin Endocrinol Metab 2014). For the other families, genome-wide sequencing of the linked short reads prepared from the parental buffy coat DNA was performed to a mean of 34-fold haploid coverage. N50 phase block length of the parental DNA samples ranged from 3 to 14 Mb with >94% of SNPs phased. N50 is an indicator of haplotyping performance and defined as the block length at which the sum of block length of that block and larger blocks represents 50% of the overall phased sequence (Snyder M W, Adey A, Kitzman J O, Shendure J., Nat Rev Genet 2015; 16:344-58; Zheng G X et al., Nat Biotechnol 2016; 34:303-11). The mean sequencing depth of maternal plasma DNA was 275-fold.

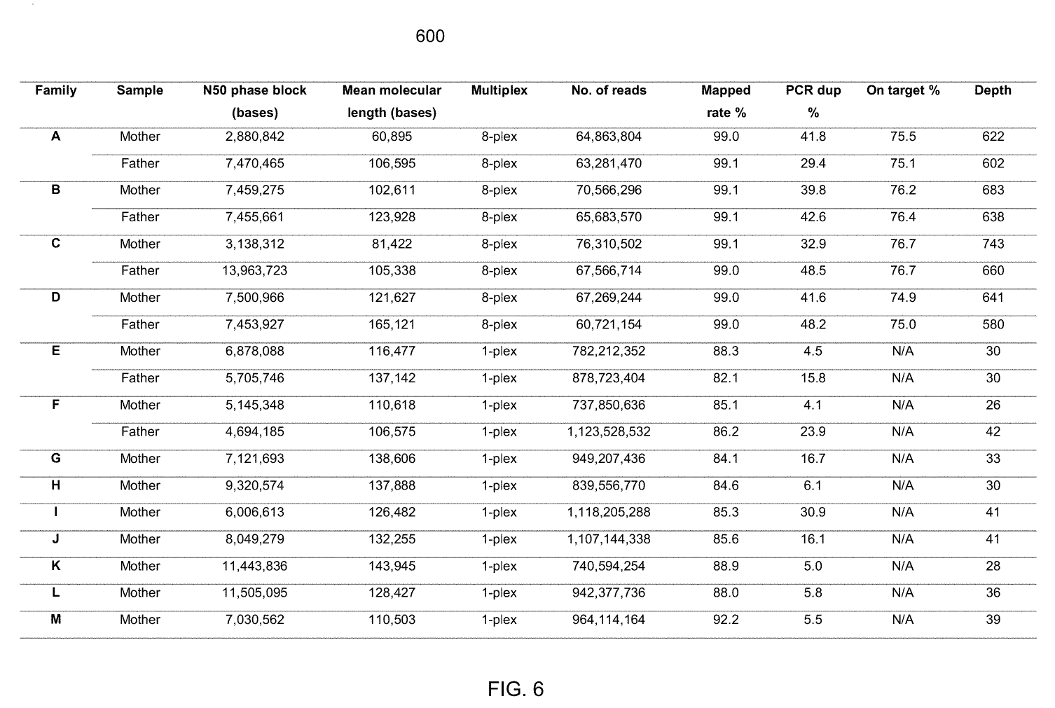

FIG. 6 shows a table 600 of sequencing data of parental genomic DNA processed with the 10×™ system according to embodiments of the present invention. N50 phase block is a statistic of a set of haplotype blocks. N50 is analogous to a mean or median of haplotype block lengths, but assigned with greater weights given to the longer haplotype blocks. The haplotype blocks phased in a sample are first ranked from longest to shortest. N50 is the length of the haplotype block with which the addition of all the blocks longer than this N50 block covered 50% of the phased sequence (e.g. 50% of the human genome in the context of whole genome haplotype or 50% of the targeted portion of the genome). Mean molecular length is the average length of the original long DNA molecule from which shorter DNA fragments with the same barcode are derived. Multiplex is the number of samples sequenced together in a sequencing reaction. No. of reads is the number of sequencing reads obtained from sequencers. Mapped rate % is the proportion of reads mapped to the human genome. The PCR dup % is the proportion of fragments sharing identical genomic coordinates for both ends that are expected to be derived from the PCR step, also called PCR duplication rate. On target % is the proportion of fragments that fell within the targeted regions as pre-designed. Depth is the average times of a nucleotide being sequenced.

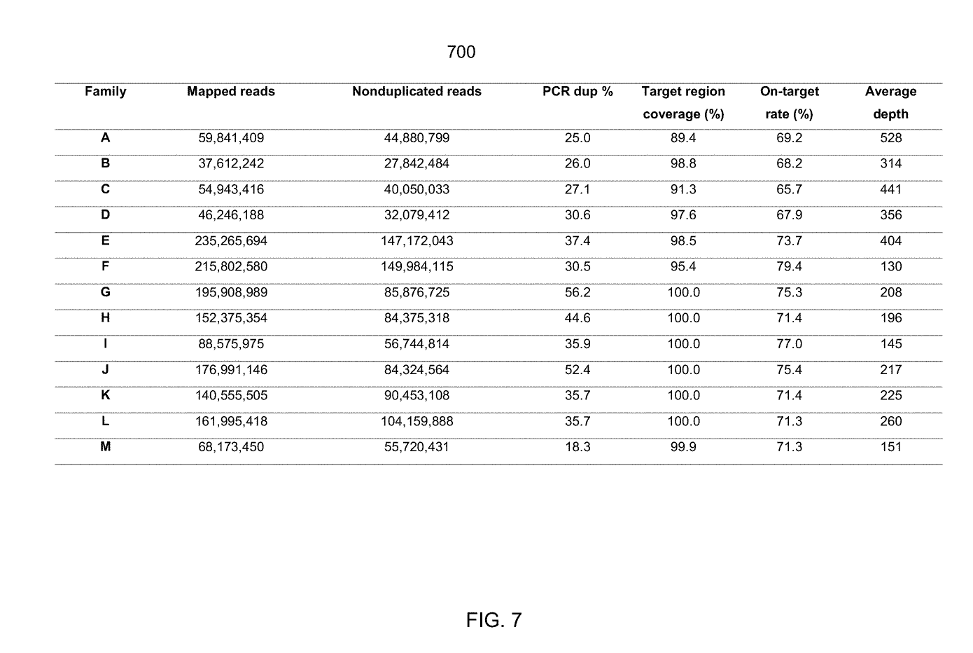

FIG. 7 is a table 700 showing an overview of targeted sequencing data of maternal plasma DNA according to embodiments of the present invention. Mapped reads is the number of sequence fragments successfully aligned to the human genome. Nonduplicated reads is the number of aligned fragments that share the identical genomic coordinate for both ends, which are all removed but one. The reads originating from the fragments after the removal of duplications with at least one distinct end are deemed nonduplicated reads. The PCR dup % is the proportion of fragments shared identical genomic coordinates for both ends that are expected to be derived from the PCR step, also called PCR duplication rate. Target region coverage is the percentage of the pre-designed regions being sequenced at least once. On-targeted rate (%) is the proportion of fragments that fell within the targeted regions as pre-designed. Depth is the average times of a nucleotide being sequenced. Average depth is the average times of a nucleotide within the pre-designed regions being sequenced.

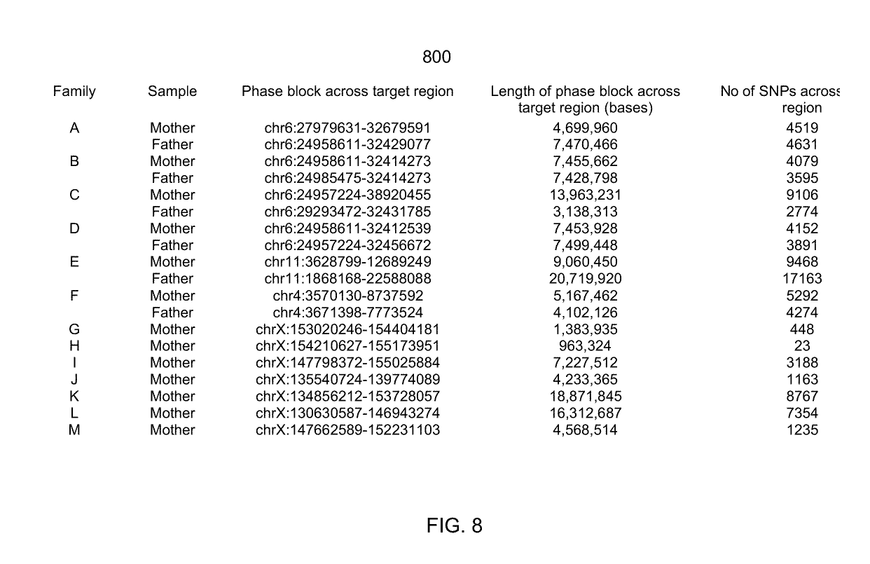

FIG. 8 shows a table 800 of haplotype phasing data for families A-M according to embodiments of the present invention. Phase block across target region is the genomic coordinates of a haplotype block spanning the targeted regions of interest, e.g., the regions containing the disease causal gene. Length of phase block across target region (bases) is the total number of nucleotides for the haplotype block spanning the target regions of interest. No. of SNP across target regions is the number of heterozygous SNPs available in the target region of interest.

1. Prenatal Assessment for Autosomal Recessive Diseases

Families A to F each presented for prenatal assessment of an autosomal recessive disease. The mutant-linked and the wildtype-linked haplotypes for the mother as well as the father were successfully determined for each of these cases, as detailed in FIG. 6. The fetal inheritance of the maternal and paternal haplotypes was determined through statistical comparisons between the maternal plasma DNA sequencing reads.

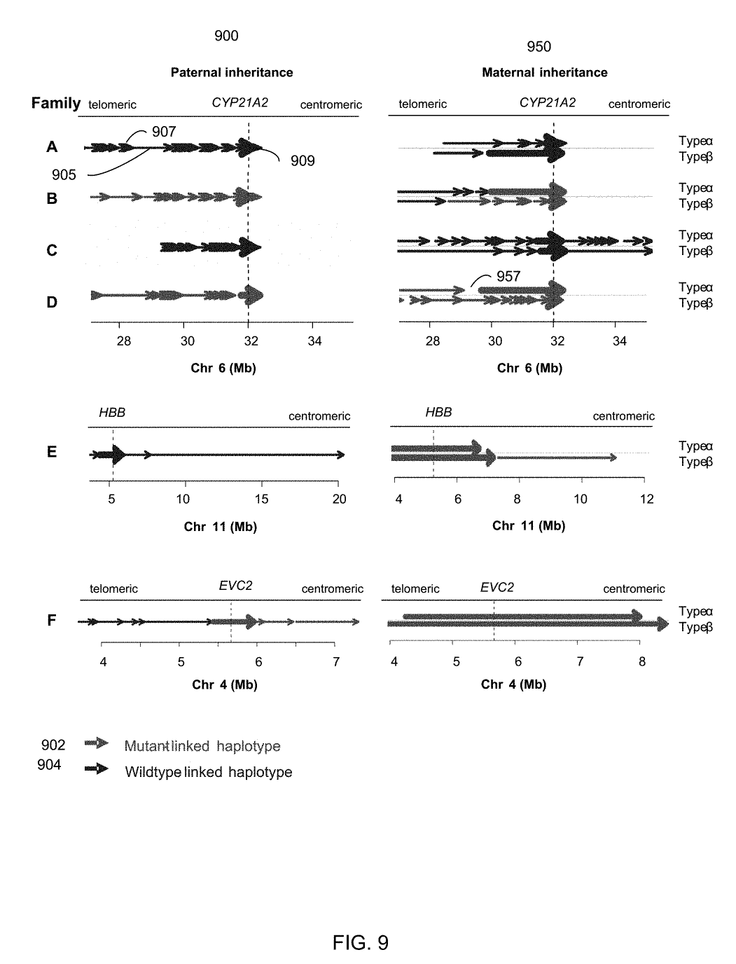

FIG. 9 shows fetal haplotype analyses in families A to F according to embodiments of the present invention. The deduced fetal genotypes were concordant with the results of the conventional diagnostic tests.

Each family has a corresponding plot with the horizontal axis being a section of the chromosome that includes the mutation. Each family has a plot for paternal inheritance and maternal inheritance. The paternal inheritance is shown in column 900, and the maternal inheritance is shown in column 950. From left to right, the horizontal axis for families A-D goes from a telomeric position to a centromeric position of chromosome 6, where the mutation is in CYP21A2 locus. For family E, the horizontal axis is from the HBB locus to a centromeric position on chromosome 11. For family F, the horizontal axis is from a telomeric position to a centromeric position on chromosome 4, where the mutation includes the EVC2 position. EVC syndrome is an autosomal recessive disease caused by mutations in the EVC or EVC2 genes and both parents were carriers for mutations on EVC2.

The analysis started from the SNPs flanking the mutation site and then extended towards the telomeric and centromeric directions. The fetal inheritance of which maternal haplotype was determined by RHDO analysis. The fetal inheritance of which paternal haplotype was determined by KS test analysis. A haplotype block is denoted by an arrow. The tail and tip of the arrow indicate the start and end positions of a haplotype block determined by a particular technique for determining a haplotype. For example, one technique is a KS test for determining paternal inheritance, and a haplotype block corresponds to a number of loci needed to make an accurate determining of haplotype inheritance. As shown, there can be many haplotype blocks in the chromosomal region for which parental haplotype information is determined.

The lengths of a string of arrows (e.g., arrow string 905) corresponds to a chromosomal region for each the parent is haplotyped. Thus, the father for family A would be haplotyped in a chromosomal region that is greater than 4 Mb. Each arrow has a different color, indicating a mutant-linked haplotype 902 (red) or a wildtype-linked haplotype 904 (blue). For example, arrows 907 and 909 correspond to the wildtype-linked haplotype for the father in family A. Arrow 909 is large to highlight the classification block across the mutation site. For the maternal inheritance, there are two arrows for each type of loci that are used: one for type α loci and the other for type β loci. For family D, there is a gap 957 between two haplotype block, resulting from a relatively long distance between two informative loci of that type, with one locus at the end of the one haplotype block and the other locus at the beginning of another haplotype block.

As an illustration, the father in family A was a carrier of a point mutation while the mother was a carrier of a 30-kb deletion at the CYP21A2 locus (as shown in table 500). Maternal blood sample was collected at the gestational age of 8 weeks and 1 day. The haplotypes of the parents were resolved from linked-read sequencing data of the parental buffy coat DNA.

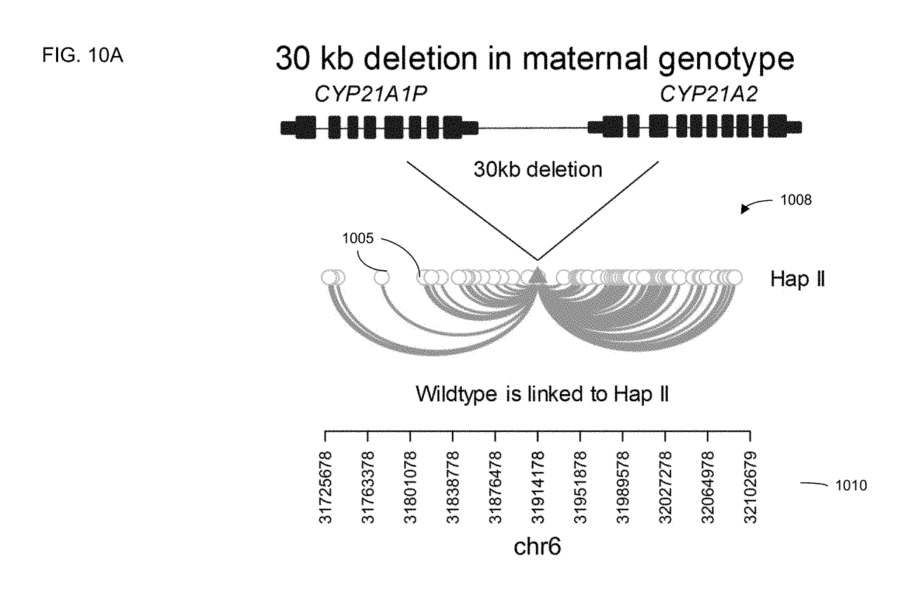

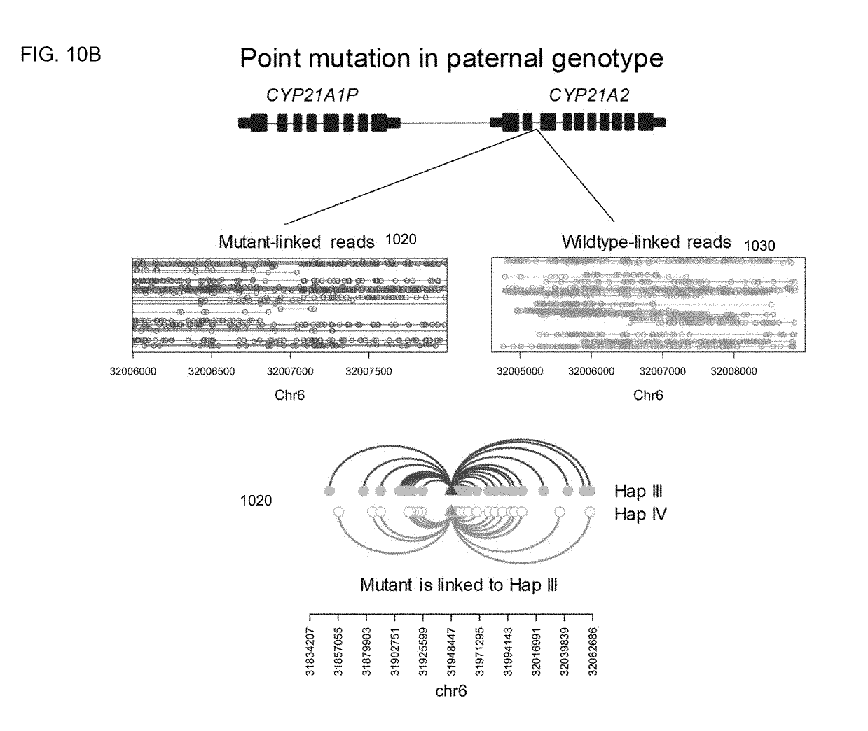

FIG. 10A shows haplotype linkage to a mutation site (30 kb deletion) for the mother in family A. The mother was a carrier of a 30 kb deletion. Heterozygous loci 1005 (specifically SNPs) were identified by aligning reads to a reference and identifying loci that had a sufficient number of reads to indicate two different alleles in the maternal genome. Sequence reads that shared the same barcode as reads with bases aligned to loci within the 30-kb deleted region were considered to be linked to Hap II, the wildtype-linked haplotype. Such reads at one of heterozygous loci 1005 were specifically considered, and the alleles on these reads were stored. Reads containing the alternative alleles (i.e., the alleles at heterozygous loci 1005 that were not linked to Hap II) were assigned to Hap I, mutant-linked haplotype. Accordingly, we inferred the other alleles as derived from the haplotype linked with the 30-kb deletion by first determining the alleles from the wildtype-linked haplotype and identifying reads having a different allele at heterozygous loci 1005 as being from the mutant-linked haplotype. The phased maternal haplotype block 1008 across the target gene was around 4.7 Mb in length and contained 4519 informative SNPs for subsequent maternal plasma RHDO analysis, as shown in table 800. The horizontal axis 1010 shows the horizontal axis across chromosome 6.

FIG. 10B shows haplotype linkage to a mutation site for the father in family A. The father was a carrier of a point mutation. The paternal point mutation was located on chr 6, at genomic coordinate 32,006,858 (GRCh37/hg19). Sequence reads 1020 that shared the same barcodes with the ones containing the paternal mutant allele were phased to one haplotype (Hap III). Sequence reads 1030 that shared the same barcodes with reads carrying wildtype alleles were phased to the opposite haplotype (Hap IV). Accordingly, alleles found on the mutant-linked reads were phased to one haplotype (Hap III), and the alleles on the wildtype-linked reads were phased to another (Hap IV). The phased haplotype block across the target gene was around 7.5 Mb in length and contained 4631 informative SNPs for subsequent maternal plasma KS test analysis, as shown in table 800.

To determine the fetal inheritance of the maternal mutations, we counted the number of plasma DNA molecules carrying informative SNP alleles. Then, we evaluated the haplotype dosage balance or imbalance of type α and type β SNPs with SPRT classification and deduced the haplotype block inherited by the fetus.

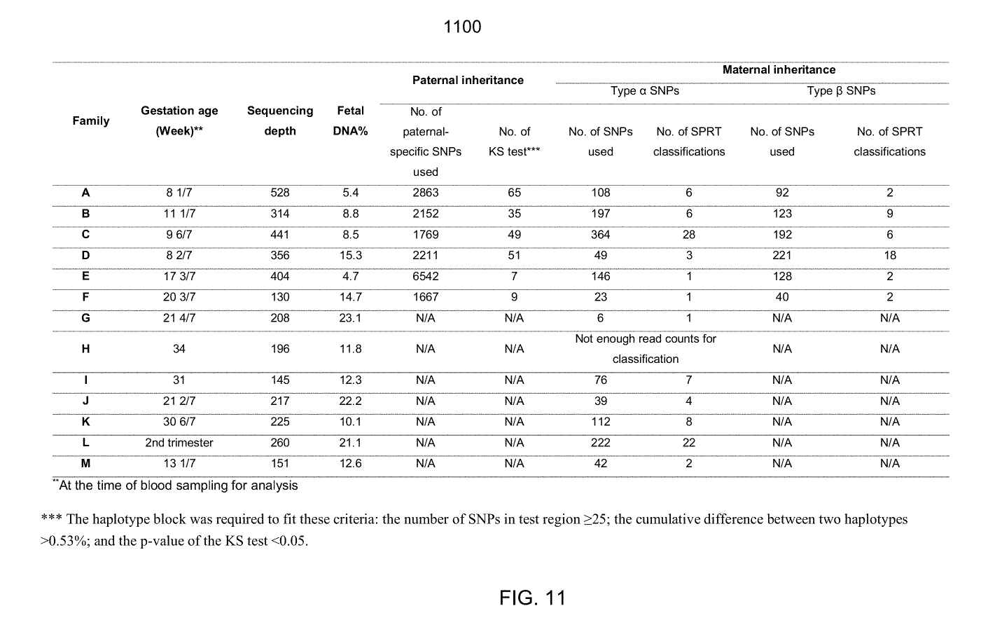

FIG. 11 is a table 1100 showing informative SNPs used for maternal plasma analysis according to embodiments of the present invention. For the maternal inheritance for family A, a total of 108 type α SNPs and 92 type β SNPs were identified and they were counted separately in the SPRT classification. For type α SNPs, an equal representation of both haplotypes was observed in 6 SPRT classifications (i.e., 6 different sets of the 108 type α loci). For type β SNPs, an overrepresentation of wildtype-linked haplotype was observed in 2 SPRT classifications (i.e., 2 different sets of the 92 type β loci). Both analyses indicated that the fetus had inherited the wildtype-linked haplotype from the mother. Only a subset of the total number of linked type α loci and 92 type β loci may be needed to accurately perform a classification of haplotype inheritance, e.g., to not be in an unclassified region.

For family A, to determine the fetal inheritance of the paternal mutation, 2863 informative SNPs within the targeted CYP21A2 region were detected in maternal plasma. 65 KS tests were done across the locus, as shown in FIG. 11. Each KS test reached statistical significance (p<0.05; minimal cumulative difference between two haplotypes >0.53%) indicating that there were more paternal-specific alleles on the wildtype-linked haplotype than those on the mutant-linked haplotype in maternal plasma. The KS test analysis supported the conclusion that the fetus had inherited the wildtype-linked haplotype from the father. We therefore concluded that the fetus did not inherit any of the parental mutations and was not affected by CAH.

The same processes were applied to families B to F and the deduced fetal genotypes and hence the disease status were concordant with the conventional prenatal diagnostic results. It is particularly noteworthy that a change in RHDO inheritance was observed in the plasma DNA data for family B and F (FIG. 9). In family B, the maternal haplotype inherited by the fetus deduced from the RHDO analysis changed from wildtype-linked to mutant-linked at around 28-30 Mb on chromosome 6. As shown in FIG. 9, the exact location of the change is different between the two types of loci used (i.e., α or β). The exact location would depend on the number of loci used in the respective haplotype blocks and the distance between neighboring loci of the two types. In family F, there is a shift in the deduced paternal haplotype inherited by the fetus from wildtype-linked to mutant-linked at around 5-5.5 Mb on chromosome 4.

In FIG. 9, a change in the color of the arrows between blue and red indicates the location where a recombination is suspected. Such a change can be detected by restarting a haplotype determination using a new set of loci once inheritance has been determined for a previous set of loci. For example, loci can be selected from a start of the chromosomal region for which parental haplotypes are known. Then, sequential loci (e.g., heterozygous loci within a specific distance of the mutation and linked to the mutation) can be used to determine a value of a property of the haplotypes until a classification can be made. Once the classification is made, a next set of loci are analyzed until another classification can be made. For families B and F, the suspected recombinations were confirmed by sequencing the chorionic villus and amniotic fluid samples, respectively.

2. Prenatal Assessment of X-Linked Diseases

Families G to L had a family history of hemophilia A or B. Family M had a family history for Hunter syndrome. Since males are hemizygous for chromosome X, only maternal haplotype analysis and fetal inheritance of the maternal X-linked mutations were performed.

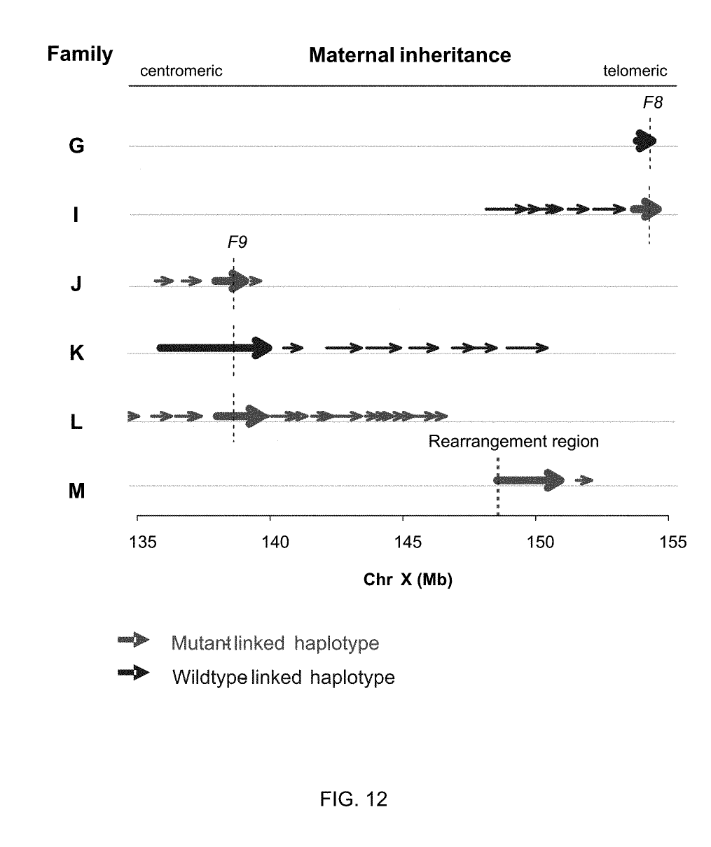

FIG. 12 shows the fetal haplotype analyses in families G to M according to embodiments of the present invention. Since males were hemizygous for chromosome X, we only analyzed maternal inheritance of the X-linked mutations. The analysis started from the SNPs flanking the mutation site and then extended towards the telomeric and centromeric directions.

As with FIG. 9, the haplotype block inherited is denoted by an arrow. The tail and tip of the arrow indicate the start and end position of a haplotype block. The fetal inheritance of which maternal haplotype was classified by RHDO analysis. A red arrow infers that an overrepresentation of mutant-linked SNP alleles in the maternal plasma DNA was classified and indicates that the fetus had inherited the mutant-linked haplotype at that locus. A blue arrow infers that an overrepresentation of wildtype-linked SNP alleles in the maternal plasma DNA was classified and indicates that the fetus had inherited the wildtype-linked haplotype at that locus.

In family G, the mother was a carrier of a point mutation on F8. Haplotypes were constructed from heterozygous SNPs on chromosome X detected from maternal genomic DNA and linkage to the mutant or wildtype allele was determined. The length of the reconstructed haplotype was 1.4 Mb and contained 448 informative SNPs for inheritance analysis. The maternal DNA was subjected to genome-wide sequencing. Due to the lower sequencing depth and problems with mapping, fewer informative SNPs were identified to construct the maternal haplotypes at the disease locus. Targeted sequencing was performed for the maternal plasma sample to provide higher sequencing depth. Due to the sparser number of informative SNPs on the phased maternal haplotypes (i.e., only 448 informative loci), only 6 of the informative SNPs were detected within the target region in maternal plasma due to difficulties in mapping. Nonetheless, one SPRT classification spanning the mutation site was achieved. The result showed an underrepresentation of informative SNPs linked with the mutant allele and indicated that the fetus had inherited the wildtype allele from the mother.

In family H, the maternal haplotype was successfully resolved via direct haplotyping. However, this particular mutation was in an SNP-depleted repeat region, and the capture probes were not specifically designed to target regions spanning this mutation site. Also, the maternal plasma volume for DNA extraction was only 0.75 mL, which was much lower than an average of 3.68 mL plasma for the other samples, and this may reduce the DNA amount for RHDO analysis. There were therefore not enough informative SNP data from the maternal plasma DNA sequencing for RHDO classification.

A recombination event was suspected from the maternal plasma DNA analysis performed for family I. The recombination was subsequently confirmed by targeted sequencing of placental DNA. Maternal haplotype analysis and maternal plasma RHDO assessment were successfully performed for families I to L. The deduced fetal genotypes were concordant with the conventional diagnostic results.

3. Direct Haplotyping of Structural Variation Using Apparent Length

In family M, the mother was heterozygous for an IDS/IDS2 gene rearrangement (translocation). IDS is normally located centromeric to IDS2 and is in the opposite orientation. Gene rearrangements in those region typically is due to intrachromosomal recombination between homologous sequences present on both IDS and IDS2 resulting in a disruption of IDS and an inversion of the intervening region. PCR amplification and restriction fragment length polymorphism analysis of maternal DNA and chorionic villi DNA identified a recombination that juxtaposed IDS intron 7 and IDS2 intron 7 (Lualdi S et al., Hum Mutat 2005; 25:491-7; Bondeson M L et al., Hum Mol Genet 1995; 4:615-21). Because of the intragenic rearrangement, there would be more short sequence reads connecting the distant genomic regions on the mutant haplotype. Thus, the paired ends of the sequenced maternal DNA molecules that contained the rearrangement would appear to be as long as HMW DNA molecules when mapped to the reference genome. We used this feature to assign SNPs to the respective haplotypes, namely SNP alleles associated with the apparently long DNA molecules were assigned to the mutant-linked haplotype. The opposite SNP alleles were then assigned to the wildtype-linked haplotype.

Accordingly, in embodiments where a long rearrangement occurs, the apparent length of a DNA fragment assembled by linking the sequence reads with the same barcode and from the same genomic region can be used for determining which haplotype is associated with the long rearrangement in a parental sample. Normally, it is known which haplotype is associated with a point mutation because there would be a sequence read covering the mutation and be eventually linked into a haplotype. But for complex rearrangements, the mutation spans a large region and is not “contained” within any one sequenced DNA molecule. In such a situation, an apparent length can be used to assign reads to a mutant haplotype. A rearrangement or other long structural variation can be identified by problems in mapping the barcoded short reads or by analyzing a coverage of the long sequence reads, as examples.

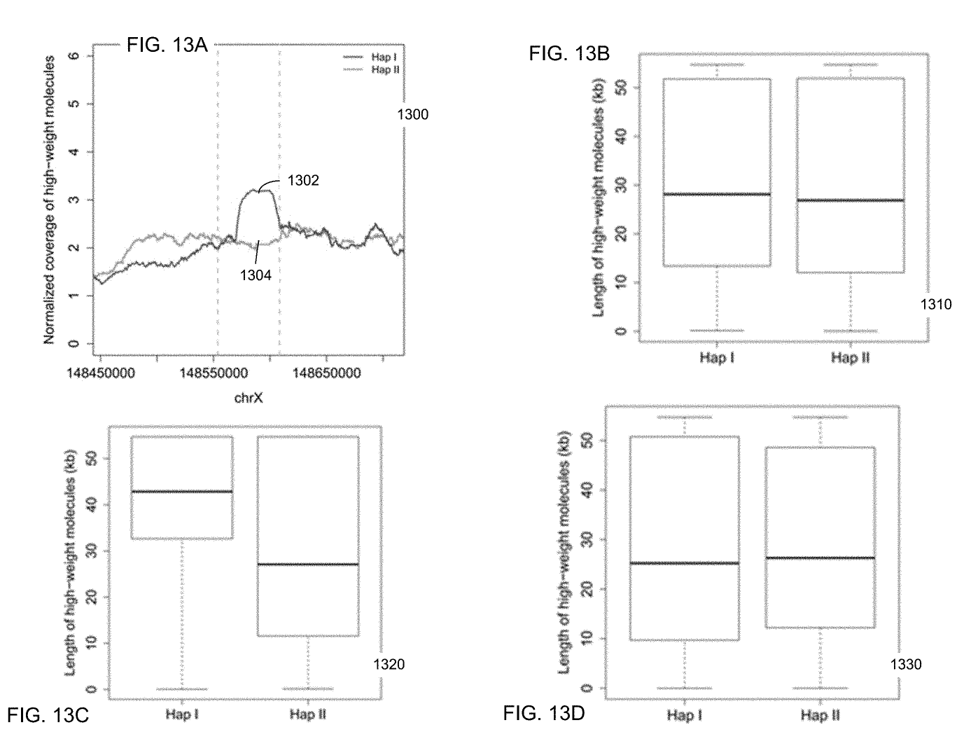

FIGS. 13A-13D illustrate haplotype assignment using linked reads obtained from the maternal genomic DNA and inferred by the increased presence of apparently long maternal DNA molecules. FIG. 13A is a plot 1300 showing the normalized coverage of linked DNA molecules with reference to total depth across chrX: 148,450,000-148,700,000 according to embodiments of the present invention. The two dashed lines indicate the location of the gene rearrangement in the IDS gene (chrX: 148,553,758-148,608,466) of the mother of family M. There is a peak observed in Hap I 1302, which represents an apparent increase in the number of long molecules that cover the region relative to Hap II 1304 and relative for other regions.

The apparent increase in long molecules covering the region is a result of the alignment artefact. Assembled linked DNA molecules contain the gene rearrangement would appear to straddle a longer distance in the reference genome. The sequenced maternal DNA molecules and assembled linked DNA were physically much smaller because the gene rearrangement results in the deletion of segment of bases between IDS and IDS2 (chrX:148553758-148608466) and the inversion would bring the more telomeric loci to a more centromeric location in the patient's genome but not in the reference genome. These apparent phenomena would then be reflected as an overrepresentation of linked DNA molecules covering the genomic locus with the gene rearrangement (FIG. 13A). It would also be reflected as the haplotype containing more longer linked DNA molecules.

The apparent increase in length of linked DNA molecules from the haplotype with the gene rearrangement is shown in the middle panel of FIG. 13B where the length of the linked or high molecular weight DNA molecules are longer on Hap I than Hap II of the genomic region with the gene rearrangement. Thus, the linked DNA molecules with relatively increased length or increased coverage can be identified as being on the same haplotype as the gene rearrangement.

FIGS. 13B-13D shows boxplots of lengths of linked DNA molecules within (plot 1320) or outside (plot 1310 or plot 1330) the gene rearrangement regions. Plot 1320 shows the distributions of lengths of long DNA molecules within the gene rearrangement regions. The average length of linked DNA molecules in Hap I was relatively longer than that in Hap II (p<0.0001). The plots 1310 and 1330 show the distributions of lengths of linked DNA molecules upstream and downstream to the rearrangement region, respectively. There was no significant difference of the lengths of linked DNA molecules between Hap I and Hap II (plot 1310: p-value=0.8665; plot 1330: p-value=0.9641). Based on these data, we inferred Hap I as the mutant-linked haplotype.

Such a technique can be used for various structural variations, such as deletions, duplications, copy-number variants, insertions, inversions and translocations (rearrangements). Besides structural variations that result in reconstructed sequence reads (i.e., long sequence reads resulting from the linked reads) that are apparently longer than average, such techniques can also be used for structural variations that result in reconstructed sequence reads that are apparently shorter than average. For example, structural variations that include large insertions or amplifications can result in reconstructed sequence reads that are shorter than average (e.g., before and after the insertion or amplification).

Accordingly when the sequencing includes linked-read sequencing of DNA molecules to reconstruct the long sequence reads from smaller linked reads, changes in the apparent length of the reconstructed long sequence reads can be used to assign sequence reads to the haplotype with the structural variation. For example, constructing the first maternal haplotype can include identifying reconstructed long sequence reads that each differ in length from an average length of reconstructed long sequence reads for regions before and after the structural variation by at least a specified length. Each reconstructed long sequence read in a region corresponding to the structural variation would differ by being smaller by a specified length or longer by a specified length. In various embodiments, the specified length can be a percentage change (e.g., 5%, 10%, 20%, 30%,40%, 50%, etc.) or an absolute length (e.g., 5 kb, 10 kb, 20 kb, 50 kb, 100 kb, or more).

Once the two haplotypes are determined based on the above length analysis, the analysis of the cell-free sample can proceed in as described herein. For example, from RHDO analysis of maternal plasma DNA, there was an overrepresentation of mutant-linked SNP alleles and this indicated that the fetus had inherited the mutant allele from the mother. The result was concordant with the clinical diagnosis and the chorionic villi analysis.

4. Discussion

Embodiments used a direct haplotyping method to resolve the parental haplotypes across disease loci, which were then used to interpret targeted sequencing data obtained from maternal plasma DNA. Using this approach, the fetal mutation profiles in 12 of 13 families, at risk for a range of single gene diseases, were successfully deduced. The mutational status of these 12 fetuses was correctly classified.

The haplotyping of the parental DNA was achieved for all 13 families. We showed that this direct whole-genome haplotyping method circumvented the need to analyze samples from related family members affected with the disease. This new development not only means that the cost of the analysis has reduced, it also means that noninvasive fetal genotyping could potentially be applied to most at-risk pregnancies.

The amount of sequence information needed can be dependent on the fetal DNA fraction, the number of loci in the selected set of loci (e.g., informative SNPs), and the sequencing depth. In the above results, we classified a sample of fractional fetal DNA concentration as low as 4.7%, with lower percentage possible with sufficient sequencing depth and not of loci. Embodiments can detect recombination, as detected in three cases in this study. A recombination event may result in incorrect fetal genotype classification if it occurs as a genomic location near the mutation. Such effects can be detected by use of apparent length of a read, as described in FIGS. 13A and 13B.

The protocol described in this study can readily be employed to many cases, e.g., with a turnaround time of about 1-2 weeks. The results demonstrate that the approach is applicable to a variety of single gene diseases. Such an approach can be universally applied as a generic protocol for the noninvasive assessment of fetal single gene diseases, thereby make noninvasive prenatal assessment of fetal single gene diseases more widely adopted. Accordingly, high-throughput linked-read sequencing followed by maternal plasma-based relative haplotype dosage analysis represents a streamlined approach for noninvasive prenatal testing of inherited single gene diseases. The approach bypasses the need for mutation-specific assays and is not dependent on the availability of DNA from other affected family members. Thus, the approach is universally applicable to pregnancies at risk for the inheritance of a single gene disease.

5. Supplemental Details

5-10 mL maternal blood samples were collected before any invasive procedures during pregnancy. Paternal and maternal blood samples were centrifuged at 1,600×g for 10 min at 4° C., and the plasma portion was re-centrifuged at 16,000×g for 10 min at 4° C. (2). Plasma, buffy coat and genomic DNA were transferred. The paternal and maternal buffy coat DNA processing and the plasma DNA processing are described in the Supplemental Methods section.

In some embodiments, the design of target capture probes for targeted sequencing can be performed in the following manner. For the prenatal assessment of congenital adrenal hyperplasia (CAH), capture probes (NimbleGen) targeting the CYP21A2 gene and the flanking regions were designed as described previously (New M I et al., J Clin Endocrinol Metab 2014; 99:E1022-30). Another set of target capture probes (NimbleGen) were designed to cover the upstream and downstream SNPs of the genes of interest including HBB (for assessment of beta-thalassemia), F8 (for hemophilia A), F9 (for hemophilia B) and IDS (for Hunter syndrome). For the prenatal assessment of Ellis-van Creveld syndrome (EVC), sequencing libraries were enriched using the SeqCap EZ Human Exome+UTR Kit (NimbleGen).

In some embodiments, paternal and maternal buffy coat DNA processing can be performed in the following manner. High molecular weight genomic DNA (HMW gDNA) was extracted from buffy coat with MagAttract BMW Kit (Qiagen). Genomic DNA was processed with GemCode™ Protocol (10×™ Genomics) for CAH cases and Chromium™ Genome Protocol (10×™ Genomics) for the other cases. The Chromium system was an upgraded version of the system that became available during the study. Long genomic DNA strands were partitioned in 10×™ barcoded gel beads. The chance that two molecules covering the same genomic locus on each gel bead is low. Barcoded oligonucleotides in a gel bead bind randomly onto the long molecules and generate short fragments with the same barcode. Libraries of the barcoded fragments were prepared and sequenced on a NextSeq500 sequencer (Illumina) with a paired-end format of 98 bp×2 (GemCode) or 150 bp×2 (Chromium) using the High Output kit (Illumina). For the CAH families, the parental genomic DNA were enriched with target capture probes before sequencing.

In some embodiments, plasma DNA processing can be performed in the following manner. Cell-free DNA was extracted from maternal plasma with the use of QIAmp DSP DNA Blood Mini Kit (QIAGEN) following the manufacturer's instructions. Libraries for maternal plasma DNA were prepared using the TruSeq Nano DNA Library Preparation Kit (Illumina) with modifications. MinElute Reaction Cleanup Kit (Qiagen) was used after end repair and adaptor ligation steps instead of magnetic bead cleanup. Elution buffer was used instead of resuspension buffer provided in the kit. The ratio of EB:LIG2:DNA adapters was adjusted to 4.17:2.5:0.83 or 3.75:2.5:1.25 depending on the input DNA amount. MinElute PCR purification Kit (Qiagen) was used after DNA enrichment instead of magnetic bead cleanup. Plasma DNA libraries were enriched with target capture probes and sequenced on the NextSeq 500 sequencer (Illumina) with a paired-end format of 75 bp×2 using the High Output kit (Illumina).

In some embodiments, sequence read alignment ca be performed in the following manner. Barcoded libraries of paternal and maternal buffy coat DNA were processed with Long Ranger pipeline provided by 10×™ Genomics. Reads that were associated with valid barcodes were aligned to the human genome (GRCh37/hg19) using the Burrows-Wheeler Aligner. Output files annotated with barcode and phasing information were generated and served as the reference haplotypes of the family for downstream analysis.

The Short Oligonucleotide Alignment Program 2 (SOAP2) was used to align the maternal plasma DNA sequence reads to the non-repeat-masked reference human genome (GRCh37/hg19) and 2 nucleotide mismatches were allowed. Duplicated reads showed identical start and end locations on the human genome were removed.

### II. Techniques Not Requiring Paternal DNA Information

In the embodiments described above, paternal genotypes (Lo Y M et al., Sci Transl Med 2010; 2:61ra91) or paternal haplotypes (New M I et al., J Clin Endocrinol Metab 2014; 99:E1022-30) were used to determine inheritance of the maternal haplotypes, e.g. for RHDO results and the description for RHSO. Specifically, the paternally-inherited allele was used to determine whether a locus was of type α or β, which impacted the classification determined from a comparison to a threshold.

However, there are circumstances where the father's DNA is not available. In this section, we develop two methods for non-invasive fetal inheritance determination that do not require the input of paternal DNA information. These approaches would render NIPT of single gene disease logistically much more practical to implement. All that is required would be a maternal blood sample. Direct haplotyping would be performed on the maternal blood cell portion and the NIPT assessment would be performed using the maternal plasma portion of the sample. Techniques that are used for RHDO and RHSO above may be used for applications here, with the selection of loci having differing criteria, and potentially the determination of a threshold differing.

A. Selecting the Set of Loci

The paternal-free techniques still determines values of a property at each haplotype, e.g., determining amounts or statistical size value of DNA fragments corresponding to each of the maternal haplotypes. But, the type of loci is not determined. The selected set of loci are heterozygous in the mother, but it is not known what the inherited paternal allele is at a given locus. Thus, no explicit deduction is made as to what paternal allele is inherited at one of the set of loci, e.g., not even using detections at other loci, as may be done using reference haplotypes, as is described in U.S. Patent Publication 2011/0105353. With a sufficient number of loci, we have identified that a specific identification of loci is not needed, e.g., when a threshold is properly selected.

The technique can be illustrated as follows. One can assume the fetus is homozygous at every maternal heterozygous SNP site within the analyzed region. If the fetus was homozygous, this would contribute to the overrepresentation of the maternal haplotype that the fetus has inherited. However, in reality, whether the fetus is homozygous or heterozygous at those maternal heterozygous SNP sites would depend on which allele the fetus has inherited from the father. As mentioned above, in the techniques of this section, we do not know the paternal genotype or paternal haplotype; and we do not attempt to deduce the paternal information, as is described in U.S. Patent Publication 2011/0105353.

If the fetus is indeed heterozygous at a maternal heterozygous SNP site (in contrast to the assumption), there would be no imbalance in allelic count at this one SNP site. It would not contribute to the statistics to help identify the maternal haplotype imbalance. However, it would generally not reverse the direction of the haplotype imbalance to cause a wrong interpretation of the fetal inheritance of the alternative haplotype because there is simply no imbalance at such a site. It is simply uninformative for the purpose of detecting the maternal haplotype imbalance. Maternal haplotype imbalance would still be detectable as long as there are sufficient SNP sites within the haplotype block to produce a statistically significant imbalance.

A difference from the technique using paternal DNA information is that the determination is which haplotype has an imbalance, whereas the analysis for type α loci and type loci was between an imbalance and a balance. The transformation of the determination to be between two different types of imbalance can enable an accurate classification without needing the paternal DNA information.

In some embodiments, loci can be selected based on population information. For example, after knowing which are the maternal heterozygous sites from the haplotyping data, one could then refer to population databases (e.g., HapMap) to identify what proportion of those SNP sites at that genomic region has a high likelihood of being homozygous. For instance, if a locus has a relatively low percentage (e.g., less than 40%, 30%, 20%, or 10%) of being heterozygous according to the population database (although heterozygous for the mother), then there can be a significant likelihood (e.g., greater than 20%, 30%, or 40%) that the fetus is homozygous. Such loci can be selected, and loci not satisfying such criteria can be discarded (i.e., not used). With a sufficient number of loci, the imbalance will be evident.

B. Determination of Maternal Inheritance from Difference in Properties

Both RHDO and RHSO can be used in this paternal-free technique. In these embodiments, maternal Hap I and Hap II can be identified by any haplotyping means, including direct methods (e.g., linked-read sequencing, single molecule sequencing, single molecule digital PCR, and other single molecule long range DNA analysis methods) and indirect methods (e.g., inference of genotype data from family based DNA analyses or statistical inference from population databases). Thus, which alleles correspond to which maternal haplotype would still be known at the set of selected loci. Embodiments are not limited to detection of a mutation, but can be used to determine an inheritance of any chromosomal region.

1. Paternal-Free Relative Haplotype Dosage Analysis (PRHDO)

The Paternal-free Relative Haplotype Dosage (PRHDO) method is based on identifying an imbalance between the two maternal haplotypes in a cell-free maternal sample (e.g., plasma). The rationale of the approach is that for any genomic loci, there are two maternal haplotypes, Hap I and Hap II. The fetus has to inherit either Hap I or Hap II. The maternal haplotype that the fetus inherits would result in an over-representation of that haplotype in maternal plasma. This haplotype imbalance could be identified among the maternal plasma DNA data by studying the accumulated allele counts of heterozygous alleles present on the respective maternal haplotypes.

When reads from the cell-free maternal sample cover the set of loci is available, a maternal haplotype imbalance can be identified by analyzing the allelic counts from those maternal heterozygous loci and summing the counts across alleles belonging to the same haplotype until an overall imbalance is detected. The haplotype that is overrepresented is the one inherited by the fetus.

To maximize the chance of detecting the imbalance with the least amount of maternal plasma DNA data, one could alter the thresholds (Zc cutoffs) to detect the imbalance based on the expected number or percentage of informative SNP sites. For example, after knowing which are the maternal heterozygous sites from the haplotyping data, one could then refer to population databases to identify what proportion of those SNP sites at that genomic region has a high likelihood of being homozygous in another person (e.g., the father and/or the fetus). The likelihood of being homozygous for a SNP locus could be deduced from the population genotypes databases, for example 1000 Genomes project or HapMap database. For each SNP, the proportion of individuals genotyped to be homozygous could be calculated, which would be deemed a likelihood of being homozygous. The cutoff used to define high likelihood of being homozygous across the haplotype block used could be, but not limited to, 70%, 75%, 80%, 85%, 90% and 95%. The absolute values of the thresholds could then be reduced based on the proportion of SNPs flagged as a high likelihood of being homozygous. For example, if 70% have a high likelihood, then the typical threshold value can be reduced by 70%.

Alternatively, after predicting which of the sites have a high likelihood of the fetus being homozygous, one could focus the allelic counting on these sites and maintain the same statistical threshold. This solution is described above in the section on selecting the set of loci. In another implementation, different weights can be assigned to the differences in allelic counts existing between two alleles derived from two haplotypes according to the probabilities of such alleles present in a population.

In setting a threshold, embodiments can account for the degree of stochastic variations in the counts of alleles at individual SNP sites due to limiting maternal DNA data at each site (to save costs). In some embodiments, the threshold values for discriminating between which maternal haplotype is inherited can be determined based on an assumed distribution, e.g., a Poisson distribution. For example, N_{hapI} and N_{HapII}, respectively corresponding to the allelic counts derived from Hap I and Hap II can be assumed to follow the Poisson distribution (Jiang, P. et al., Bioinformatics 28, 2883-2890, doi:10.1093/bioinformatics/bts549).

N_{hapI}˜Poisson(λ₁)

N_{hapII}˜Poisson(λ₂)

The fetal DNA fraction is assumed to be f and the total accumulated DNA fragments from Hap I and Hap II is assumed to be N. It is expected that there is no net dosage imbalance between the maternal heterozygous alleles when the sample does not contain any fetal DNA. Therefore, allelic counts of maternal Hap I or Hap II is assumed to be N*0.5 when f is 0. When the sample contains fetal DNA, it can be assumed that the fetus is homozygous at all the analyzed maternal heterozygous SNP sites. If the fetus inherits the maternal Hap I, then λ₁ would be N*(0.5+f/2) and λ₂would be N*(0.5−f/2). N_{hapI}−N_{hapII} approximately follows the normal distribution with the mean of N*f and the standard deviation of \sqrt{N}. The degree of the allelic count differences between the maternal Hap I and Hap II can be measured in terms of z-score by:

$$Zc = \frac{{N}_{Hap I} - {N}_{Hap II}}{\sqrt{N}} \qquad ( 1 )$$

If Zc is above 3, the fetus would inherit the Hap I; if Zc is below −3, the fetus would inherit the Hap II. The fetus must inherit either haplotype I or II from the mother. Therefore, when Zc is <3 but >−3, it would mean that there is inadequate statistical evidence, for example, inadequate number of sequenced reads or fetal DNA fraction, to make a determination of the fetal inheritance of that region. In that case, additional loci in the set can be tested for a particular haplotype block, as long as more heterozygous loci are available. More loci may not always be available, e.g., when a particular mutation is to be detected and the loci are required to be within a specified distance of the mutation.

Accordingly, Poisson statistics (or other statistics) can be used to capture such variations and set cutoffs that would identify the haplotype imbalance and allelic skewing beyond that accountable by stochastic variation. Other statistics, for example but not limited to binomial distribution, normal distribution, gamma distribution, Beta distribution, negative binomial distribution, Hidden Markov model, Monte Carlo simulation, and expectation—maximization algorithm, as well as machine learning algorithm, can be also used to capture such variations.

The maternal cell-free sample can be analyzed in various way. As examples, the maternal plasma DNA data could be obtained by whole genome sequencing, by targeting the genomic regions of interest, or by multiple digital PCR assays to provide allelic accounts across individual SNP sites, or similarly by microarray or mass spectrometry or other quantitative methods to determine the allelic ratios of SNPs within the haplotype. Both maternal and fetal DNA molecules in plasma are short fragments or just several hundred bases long. Thus, the sequencing, digital PCR or other quantitative allelic ratio measurements in maternal plasma are based on individual SNPs. But the statistical interpretation of the haplotype imbalance can use the collective allelic counts of multiple informative SNPs along the haplotype block using the maternal Hap I and Hap II as scaffolds.

If the mother is a carrier of a mutation for a genetic disease, one would be able to identify from the maternal haplotype information whether Hap I or Hap II contains the maternal mutation. After performing PRHDO, embodiments can determine which maternal haplotype the fetus has inherited and whether it is the haplotype associated with the maternal mutation. If yes, the fetus is deemed to have inherited the maternal mutation. To determine the paternal mutation or paternal haplotype, one could then search for mutant and wildtype alleles present in maternal plasma but not present in the maternal haplotypes. These are typically SNP sites where the mother is homozygous and the fetus has inherited a different allele. If the paternal mutation is different from the maternal mutation, such non-maternal mutation could be identified from the maternal plasma DNA data quite readily as a qualitatively different sequence. In such a context, no paternal genetic or genomic information is needed. Thus, whether PRHDO is used for determining the fetal genetic or genomic information or mutational status, no paternal information would be needed.

2. Paternal-Free PRHSO

Size can be used in a similar manner as count-based techniques. For example, one threshold can be used to detect whether a first maternal haplotype is inherited, and a second threshold can be used to detect whether a second maternal haplotype is inherited. Additionally, paternal-free relative haplotype-based size shortening analysis (PRHSO) can select loci as described above.

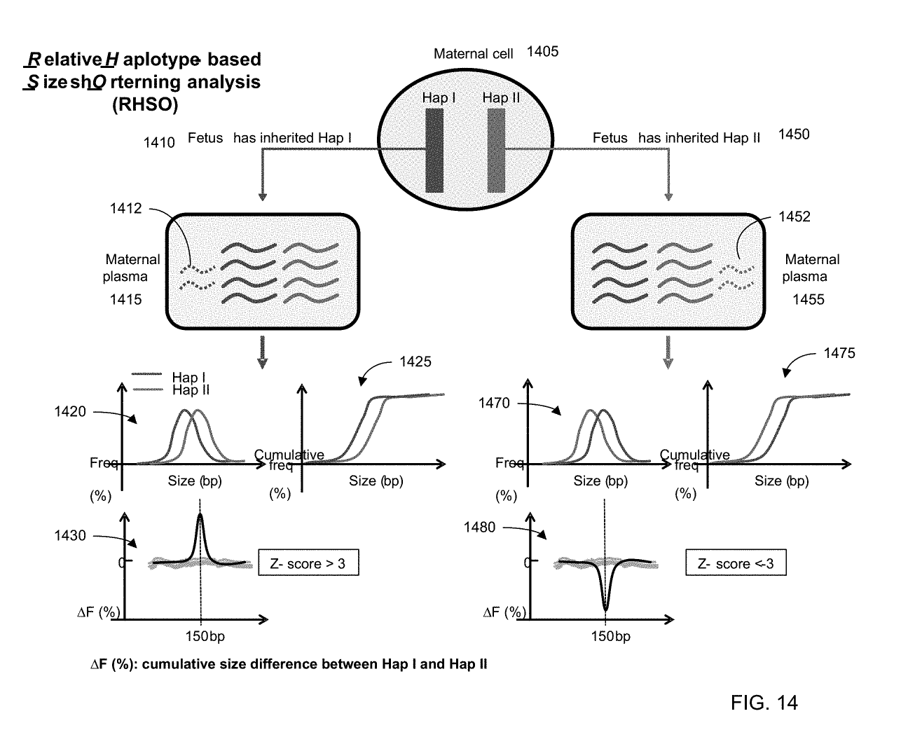

FIG. 14 is a schematic illustration of a paternal-free RHSO principle according to embodiments of the present invention. FIG. 14 shows cellular DNA 1405 (i.e., from cellular tissue), which can be used to determine the maternal haplotypes. To obtain the two haplotypes (i.e. Hap I and Hap II), cellular DNA from maternal cell 1405 can be analyzed using direct haplotyping techniques, such as microfluidics-based linked-read sequencing (Zheng G X et al., Nat Biotechnol 2016; 34:303-11). As examples, the cellular DNA can be obtained from blood cell DNA obtained from the pregnant woman or deduced from parent-offspring trio genotypes (New M I et al., J Clin Endocrinol Metab 2014; 99:E1022-30). The SNPs linked to the disease-causing gene were assigned as Hap I.

FIG. 14 shows two branches. Branch 1410 corresponds to an analysis and results that would occur if the fetus inherited Hap I. Branch 1450 corresponds to an analysis and results that would occur if the fetus inherited Hap II.

If the fetus has inherited Hap I (branch 1410), more fragments carrying alleles of Hap I are present in maternal plasma 1415 in comparison with those carrying alleles of Hap II. The shorter DNA fragments 1412 derived from the fetus cause the DNA fragments of Hap I to collectively be shorter than the DNA fragments of Hap II. Plot 1420 shows a size distribution for Hap I and a size distribution for Hap II. As shown, the size distribution for Hap I is shifted to the left (i.e., to smaller sizes) relative to the size distribution for Hap II. This shift to smaller DNA fragments is a result of fetal DNA fragments 1412.

Plot 1425 shows the cumulative size distribution as determined from plot 1420. The cumulative distribution is a plot of the area under the curves in plot 1420 at each size. The cumulative distribution increases most rapidly when at a peak in the corresponding size distribution. The fetal DNA fragments 1412 also shift the cumulative size distribution of Hap I towards the shorter end compared to that of Hap II.

To quantify the degree of size shortening of Hap I, the difference in cumulative size frequencies (ΔF) for size profiles between Hap I and Hap II was constructed, as shown in plot 1430. In other words, the progressive accumulation of plasma DNA molecules, from short to long sizes, as a proportion of total plasma DNA molecules in a sample was determined on the basis of the maternal Hap I and Hap II. The difference between the two curves ΔF was then calculated as follows:

ΔF=S_{Hap I}−S_{Hap II} (2)

where ΔF represents the difference in the cumulative frequencies between the maternal Hap I and Hap II at a particular size, and S_{Hap I} and S_{Hap II} represent the proportions of plasma DNA fragments less than a particular size from the maternal Hap I and Hap II, respectively. A positive value of ΔF for a particular size suggests a higher abundance of DNA shorter at that particular size on the maternal Hap I compared with the Hap II. ΔF is an example of a separation value.

A threshold can be used to determine whether ΔF is sufficiently large to make an accurate determination of the inherited haplotype. In FIG. 14, the threshold is identified as being greater than 3 when the separation value is determined as a z-score, which accounts for typical variations, e.g., a standard deviation. A threshold can be considered sufficiently large when the maternal plasma DNA measurement is a specified number (e.g., 2 or 3) of standard deviations away from reference data that captures the stochastic variation of cumulative size measurement in maternal plasma. A set of reference data could be simulated. For example, under an assumption of no size difference between the DNA molecules from Hap I and Hap II, random permutations of the two groups of DNA molecules derived from Hap I and Hap II were generated 30 times. During these permutations, the phase information was not taken into account. Thus, the permuted results would represent the background stochastic variation. For each permutation, the differences in the cumulative frequencies (ΔF) between the simulated Hap I and Hap II were calculated and expected to be zero. In an embodiment that uses a ratio, the expected value can be one. To statistically quantify the degree of size difference between maternally transmitted and untransmitted haplotypes in maternal plasma, the extent of size difference at a particular size in a testing sample was calculated by using z-score. Z-score (e.g., equation (3) below) can be calculated by comparing the ΔF₁₅₀ (the ΔF of the testing sample at size 150 bp) deduced from the real test data with the mean and standard deviation for ΔF derived from the simulated reference data at 150 bp. Theoretically, M is expected to be 0. If Zs is greater than 3, it will suggest the fetal inheritance of Hap I. If Zs is less than −3, it will suggest the fetal inheritance of Hap II.

If the fetus has inherited Hap II (branch 1450), more fragments carrying alleles of Hap II are present in maternal plasma 1455 in comparison with those carrying alleles of Hap I. The shorter DNA fragments 1452 derived from the fetus cause the DNA fragments of Hap II to collectively be shorter than the DNA fragments of Hap I. Plot 1470 shows a size distribution for Hap I and a size distribution for Hap II. As shown, the size distribution for Hap II is shifted to the left (i.e., to smaller sizes) relative to the size distribution for Hap I. This shift to smaller DNA fragments is a result of fetal DNA fragments 1452.

Plot 1475 shows the cumulative size distribution as determined from plot 1470. The cumulative distribution is a plot of the area under the curves in plot 1470 at each size. The fetal DNA fragments 1452 also shift the cumulative size distribution of Hap II towards the shorter end compared to that of Hap I. Plot 1430 shows ΔF being negative since S_{Hap II} increases earlier than S_{Hap I}.

Accordingly, if the fetus has inherited Hap I from the mother, Hap I is overrepresented in maternal plasma. Since the fetal-derived Hap I plasma DNA are shorter, the size profile of Hap I would be shifted to the left hand with respect to that of Hap II, resulting in an increase in the cumulative size difference between Hap I and Hap II (ΔF) at 150 bp. Conversely, if the fetus has inherited Hap II from mother, the resultant ΔF at 150 bp would give a negative value.

Other statistical values of a size distribution may be used besides a proportion of plasma DNA fragments less than a particular size from a maternal haplotype. Other examples are provided herein. For example, a ratio of a number of DNA fragments from one size range relative to a number of DNA fragments in a different size range may be used. The two size ranges may overlap, but have at least a start and end of the range that is different.

C. Results

We retrieved data of the 27 cases from a previous study (New M I et al., J Clin Endocrinol Metab 2014; 99:E1022-30) and the data used for section I. Targeted massively parallel sequencing was performed on maternal, paternal, and proband's genomic DNA in each family to detect respective genotypes and to deduce the parental haplotypes in New et al. study. Microfluidic-based linked-read sequencing (10× Genomics) on maternal genomic DNA was carried out for haplotype phasing, as described in section I. Maternal plasma DNA were subjected to targeted sequencing in all samples with different sets of capture probes (NimbleGen). Each library was sequenced on a HiSeq 2000 (Illumina) or HiSeq 1500 (Illumina) or NextSeq 500 sequencer (Illumina) with a paired-end format. The sequencing data were aligned on Short Oligonucleotide Alignment Program 2 (SOAP2) or Long Ranger pipeline provided by 10×™ Genomics.

From the 27 cases we analyzed, the call rate of RHSO analysis was 74.1% with 100% accuracy. The higher the fetal fraction and size difference between the two maternal haplotypes, the lower the number of DNA molecules was required for successful classification. We demonstrate that a size-based approach is feasible as an independent assay to test and validate the fetal inheritance of single gene mutations in a non-invasive fashion without the need of paternal genotype information.

1. Degree of Size Difference between Hap I and Hap II

We analyzed the size distribution of DNA fragments carrying Hap I and Hap II alleles respectively. The size of each plasma DNA molecule was deduced from the genomic coordinates of the ends of the pair-ended sequenced reads. To determine the size of a plasma DNA molecule, one could either sequence through the entire molecule either by massively parallel sequencing, such as with the use of sequencing-by-synthesis methods, semiconductor sequencing, or single molecule sequencing, such as by the Oxford nanopore system or Pacific Biosciences system.

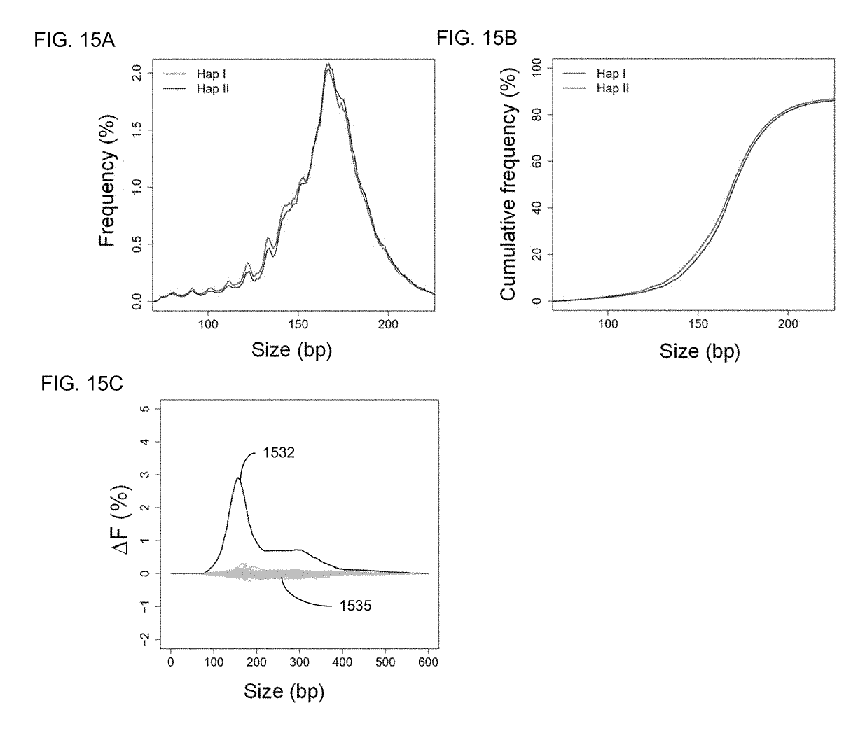

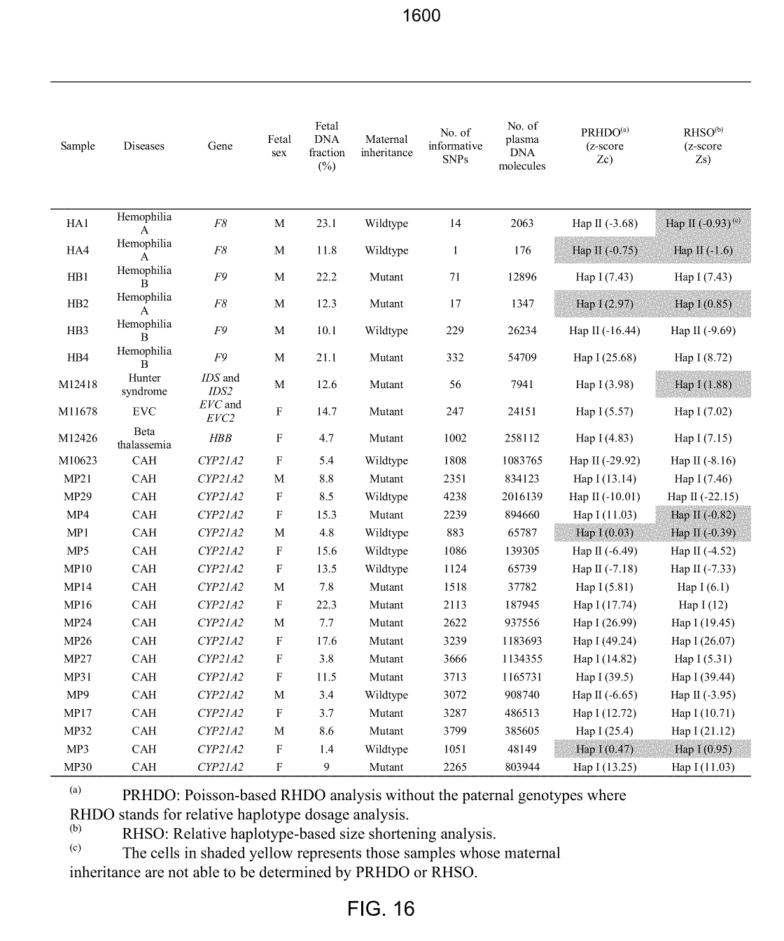

FIGS. 15A-15C show representative size profiles between Hap I and Hap II according to embodiments of the present invention. The data correspond to case MP31, whose data is shown in table 1600 of FIG. 16.

FIG. 15A shows a frequency distribution plot of the abundance of maternal plasma DNA molecules of various sizes that were associated with Hap I or Hap II. There was a higher proportion of DNA molecules from Hap I than Hap II at the sizes ranged from 100 to 150 bp.

FIG. 15B shows cumulative frequencies of the size distribution of maternal plasma DNA molecules associated with Hap I or Hap II. The cumulative frequency curve of DNA molecules from Hap I shifted relatively towards the left of that of Hap II.

FIG. 15C shows the cumulative difference between the size distribution of maternal plasma DNA molecules associated with Hap I and Hap II. In this example, ΔF between the maternal Hap I and Hap II at a particular size was calculated for each case. The ΔF at the size of 150 bp was approximately the maximum 1532 in FIG. 15C. Therefore, 150 bp was chosen as a cut-off value to statistically quantify the degree of size difference.

The gray lines 1535 were generated from simulated data under the assumption of no size difference between the DNA molecules from Hap I and Hap II. The set of simulated reference data under an assumption in which there was no size difference between the DNA molecules from Hap I and Hap II was generated by randomly permuting the two phases of maternal haplotypes 30 times. The differences in the cumulative frequencies (ΔF) between the simulated Hap I and Hap II were calculated and expected to be zero.

To statistically quantify the degree of size difference between maternally transmitted and untransmitted haplotypes in maternal plasma, the extent of size difference at a particular size in a testing sample was calculated by comparing with simulated reference data using the below formula in the format of z-score (Zs):

$$Zs = \frac{Δ {F}_{150} - M}{SD} \qquad ( 3 )$$

where ΔF₁₅₀ represented the ΔF of the testing sample at size 150 bp; and M and SD represented the mean and standard deviation for ΔF derived from the simulated reference data at 150 bp. Theoretically, M is expected to be 0. If Zs is greater than 3, Hap I is suggested to be transmitted to the fetus. If Zs is less than −3, Hap II is suggested to be transmitted to the fetus. Zs is another example of a separation value, or alternatively a way to specify a threshold.

In the case MP31, Zs is 39.44 (Table 1600), which is greater than 3. Therefore, it suggested that the fetus had inherited the maternal Hap I. The result was concordant with the clinical diagnosis.

2. PRHSO Performance

FIG. 16 is a table 1600 showing a summary of PRHSO and PRHDO performance according to embodiments of the present invention. We analyzed two experimental datasets mentioned above to evaluate the sensitivity and specificity of PRHSO. The two datasets were different by the haplotype phasing method. New et al. inferred the maternal haplotypes by genotyping maternal, paternal and proband's DNA. Section I applied the microfluidic-based linked-read sequencing to directly phase the maternal haplotypes. In total, we tested 27 families at risk of an autosomal recessive disease or X-linked disease, including congenital adrenal hyperplasia (CAH), Ellis-van Creveld syndrome (EVC), beta thalassemia, hemophilia and Hunter syndrome. The mean sequencing depth of the maternal plasma ranged from 25 to 528 fold (median: 217 fold) haploid human coverage. The fetal DNA fraction in maternal plasma ranged from 1.4% to 23.1% (median: 10.1%).

Using RHSO method, 20 out of 27 (74.1%) cases were classified. The maternal inheritance status of these 20 cases was correctly deduced. For the remaining 7 cases, Zs was between 3 and −3 and thus no classification of fetal inheritance was made.

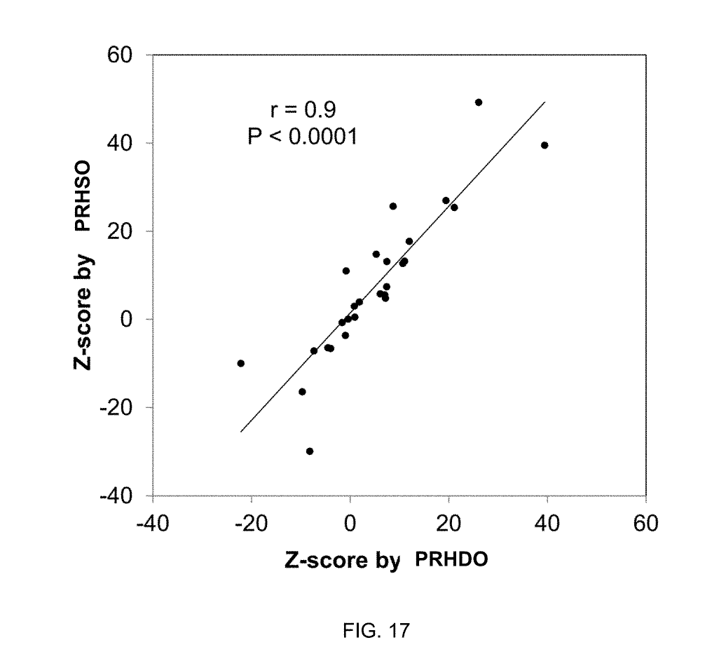

FIG. 17 shows the correlation of the degree of imbalance between Hap I and Hap II reflected in size- and count-based analysis according to embodiments of the present invention. To compare the RHSO performance with the count-based approach without requiring the information of paternal haplotypes (PRHDO) was used to measure the dosage imbalance of the alleles on each of the two maternal haplotypes regardless of molecular size (Lo Y M et al., Sci Transl Med 2010; 2:61ra91; Fan, H. C. et al. Nature 487, 320-324, doi:10.1038/nature11251, 2012).

The call rate for PRHDO was 85.2% compared to 74.1% for RHSO. Both classifications had 100% accuracy. No fetal inheritance was made for cases with Zc between 3 and −3. The magnitudes of molecular imbalance between maternal Hap I and Hap II present in maternal plasma sample were concordantly (Pearson's r=0.9, p-value<0.0001) reflected by RHSO and PRHDO analyses

Accordingly. we have demonstrated the feasibility of paternal-free Relative Haplotype-based Size shOrtening (PRHSO), to infer the maternal inheritance of the fetus from sequencing data of cell-free DNA in maternal plasma. This method was based on calculating the size difference between maternal haplotypes. Using PRHSO, in 27 families at risk of a range of single gene diseases, 20 fetal mutational profiles were correctly classified.

3. Minimal Number of Molecules Required for PRHSO and PRHDO

We also investigated the minimal number of plasma DNA molecules required for PRHSO or PRHDO classification using computer simulation. Case MP16 was selected for a model dataset since this case had adequate fetal DNA fraction and enough SNP sites for downstream data analysis. We first separated the fetal and maternal plasma DNA size profiles by examining the fetal-specific and maternal-specific DNA fragments respectively. The SNP loci where the mother was homozygous and the fetus was heterozygous were used to deduce the fetal-specific alleles. On the other hand, the SNP loci where the mother was heterozygous and the fetus was homozygous were used to deduce the maternal-specific alleles. With reference to the fetal and maternal plasma DNA size profiles, we could in silico simulate different numbers of DNA molecules derived from maternal Hap I and Hap II, which were contributed by both the mother and the fetus, by varying sequencing depths, fetal DNA fractions and plasma DNA sizes by computationally include different plasma DNA species (maternal or fetal, short or long) from the MP16 dataset into the simulated sample dataset.

Fetal DNA fraction is one of the factors that can affect the number of DNA molecules required for analysis. Under a certain fetal DNA fraction, we examined the total number of DNA fragments required for PRHSO to reach 95% sensitivity using Zs >3 with the use of the model dataset. As a comparison, the total number of DNA fragments for PRHDO at 95% sensitivity was also determined.

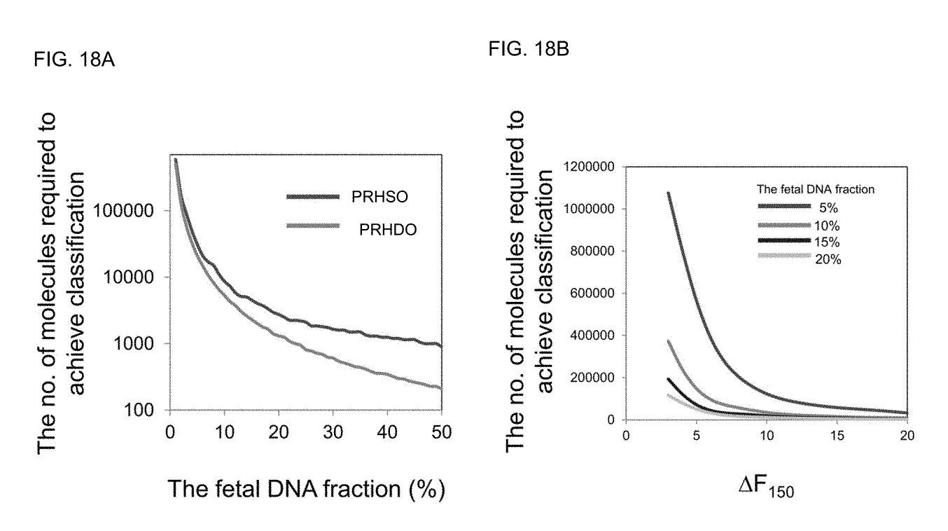

FIG. 18A shows the number of analyzed plasma DNA molecules per haplotype block required to achieve classification as a function of varying fetal DNA fraction in the maternal plasma sample. The number of molecules required is exponentially reduced as the fetal DNA fraction increases for both PRHSO and PRHDO. At the same fetal DNA fraction, the number of molecules needed for PRHDO classification was lower than that of PRHSO. PRHSO required relatively more molecules to obtain the same level of accuracy compared with PRHDO. This explained why there were more unclassified cases by PRHSO than PRHDO analysis.

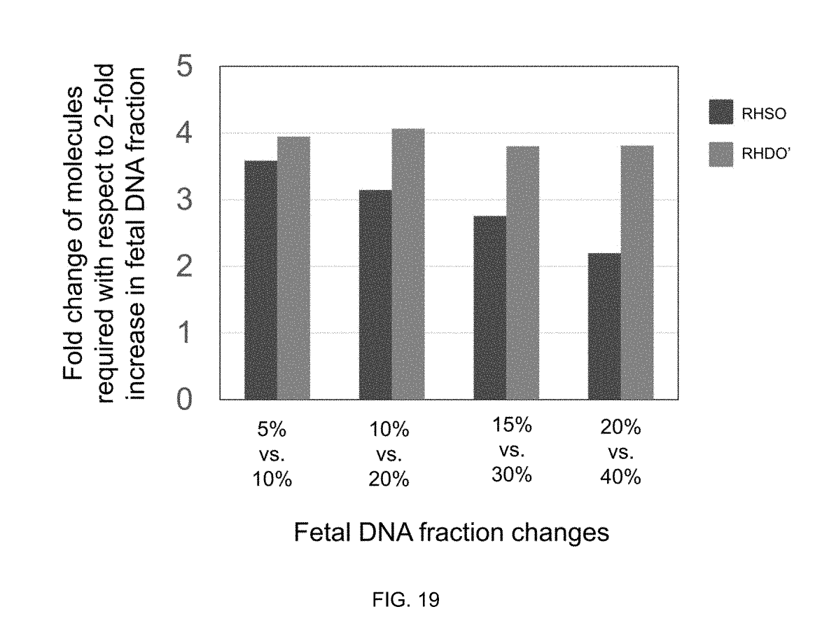

FIG. 19 shows the fold change in the number of plasma DNA molecules required for haplotype block classification when the fetal DNA fraction in the sample is doubled from 5%, 10%, 15%, or 20% according to embodiments of the present invention. When there was a 2-fold reduction in fetal DNA fraction, the number of DNA molecules required for PRHDO would be quadrupled while the fold-increase in the number of DNA molecules required for RHSO was less.

Besides the fetal DNA fraction, another factor that can affect the number of DNA molecules required to make a classification with 95% sensitivity using RHSO was the difference of size distribution between maternally-derived DNA and fetally-derived DNA in maternal plasma. To understand the relationship of the size distribution difference and the number of molecules required, we simulated a range of cumulative size differences between maternal Hap I and Hap II at a size of 150 bp (ΔF₁₅₀) from 1% to 20%. We then calculated the number of DNA molecules required at fetal DNA fraction of 5%, 10%, 15% and 20%, respectively.

FIG. 18B shows the number of analyzed plasma DNA molecules per haplotype block required to achieve classification as a function of varying degree of size differences (ΔF) between fetal and maternal DNA as well as varying fetal DNA fraction in the maternal plasma sample. FIG. 18B shows the theoretical number of molecules required to reach a sensitivity of 95% at a given fetal fraction and ΔF₁₅₀. Under a particular fetal DNA fraction, when there is a 2-fold reduction in ΔF₁₅₀, the number of DNA molecules required to be analyzed would be approximately quadrupled.

According to this computer simulation analysis, given the fetal DNA of 5%, the sequencing depth of 100 fold and the cumulative size difference between the maternal and fetal DNA molecules generally greater than 20% at 150 bp, the minimal number of SNPs required would be 310. With reference to the simulation results, the cases that were unclassified can be explained by the insufficient number of molecules being analyzed for PRHSO or PRHDO calculation.

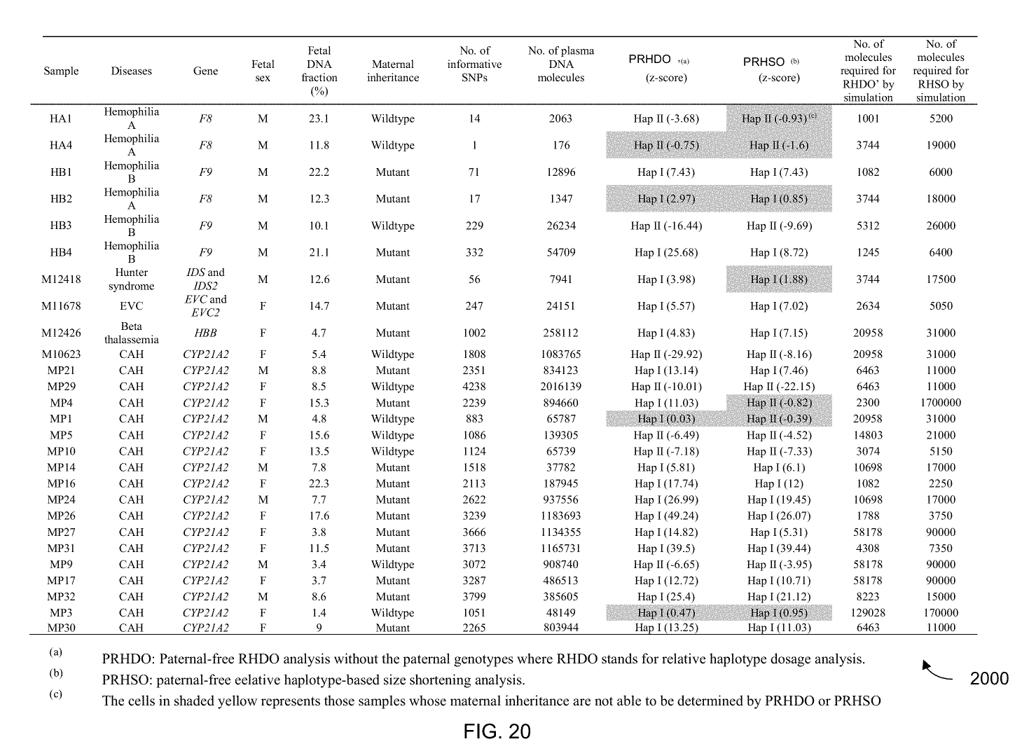

FIG. 20 is a table 2000 showing the theoretical number of molecules required in PRHSO and PRHDO analysis for the real cases according to embodiments of the present invention. Table 2000 shows that the majority of the unclassified cases did not have enough DNA molecules analyzed as predicted by the simulation based on the fetal DNA fraction and DNA size difference of that sample. Thus, increased amount of analysis from the sample, for example by increasing the sequencing depth, or by collecting a sample later in gestational age where the fetal DNA fraction might become higher, may allow the fetal inheritance to become classifiable. The cells in shaded yellow represents those samples whose maternal inheritance are not able to be determined by PRHDO or RHSO

The computer simulation for RHSO and PRHDO were conducted by R scripts (www.r-project.org). For RHSO simulation analysis, the fetal DNA fraction is assumed to be f. It is assumed that the heterozygous alleles in maternal DNA are analyzed. “rbinom” function in R program was used to simulate the plasma DNA molecules derived from maternal Hap I and Hap II according to the expected fractions of μ₁ and μ₂, respectively. If the fetus inherits the maternal Hap I, then μ₁ is (0.5+f/2) and μ₂ is (0.5−f/2). The fetal and maternal DNA sizes in maternal plasma were simulated according to the empirical size distributions of fetal and maternal DNA molecules, respectively. “sample” function in R program was used to randomly sample the sizes (simulated dataset A) comprising the fetal and maternal DNA sizes for maternal Hap I and Hap II based on the corresponding amount of plasma DNA molecules determined by the aforementioned “rbinom” function. On the other hand, dataset B in which no dosage imbalance is assumed to be present between maternal Hap I and Hap II in maternal plasma is simulated by assign the μ₁ and μ₂ with 0.5. ΔF₁₅₀ were determined in simulated dataset A and B. ΔF₁₅₀ in simulated dataset B was used to create the M and SD in the formula (3). Thus, for dataset A, we can calculate the z-score in RHSO simulation analysis. For PRHDO simulation analysis, we can directly apply the “rbinom” function with μ₁ and μ₂ to simulate the allelic imbalance present between maternal Hap I and Hap II in plasma. Afterward, formula (1) was used to calculate the z-score in PRHDO simulation analysis.

4. Use of Sliding Window to Detect

Accurate fetal haplotype determination can also depend on accurate detection of recombination, namely where the inherited fetal haplotype switches between Hap I and Hap II. For example, embodiments could identify recombinations by either analyzing discrete-sized haplotype blocks and interpret one block at a time. Alternatively, one could use a sliding window approach to determine which haplotype the fetus has inherited within smaller genomic regions and continue to lengthen the region as long as the haplotype imbalance in maternal plasma still points to the same haplotype. For example, a 200 kb sliding window could be used to analyze the haplotype block dosage imbalance using the aforementioned formula (1). A 200 kb window is expected to have 200 SNPs (1 SNP per kilobase). Therefore, 50 heterozygous SNP sites would be analyzed assuming that the average heterozygosity rate is 25%.

According to FIG. 18A, 20,000 molecules (i.e. 400× coverage per SNP) would allow classification of haplotype block inheritances in a pregnancy with a fetal DNA fraction of 5%. If we detect a classification change between two consecutive sliding windows (or other number of consecutive windows, e.g., 3, 4, etc.), it would suggest a recombination present in between such two consecutive haplotype blocks. The other window sizes including but not limited to 5 kb, 10 kb, 20 kb, 30 kb, 40 kb, 50 kb, 60 kb, 70 kb, 80 kb, 90 kb, 100 kb, 300 kb, 400 kb, 500 kb can be used. The choice of window size can be adjusted according to the number of actual SNPs being analyzed, the sequencing depth achieved as well as the recombination rate. The higher sequencing depth and higher density of SNPs would lead to the smaller the window size required; and thus the higher resolution in detecting the recombination can be achieved. The lengthening of the block can stop whenever a next region suggests the alternative haplotype showed imbalance.

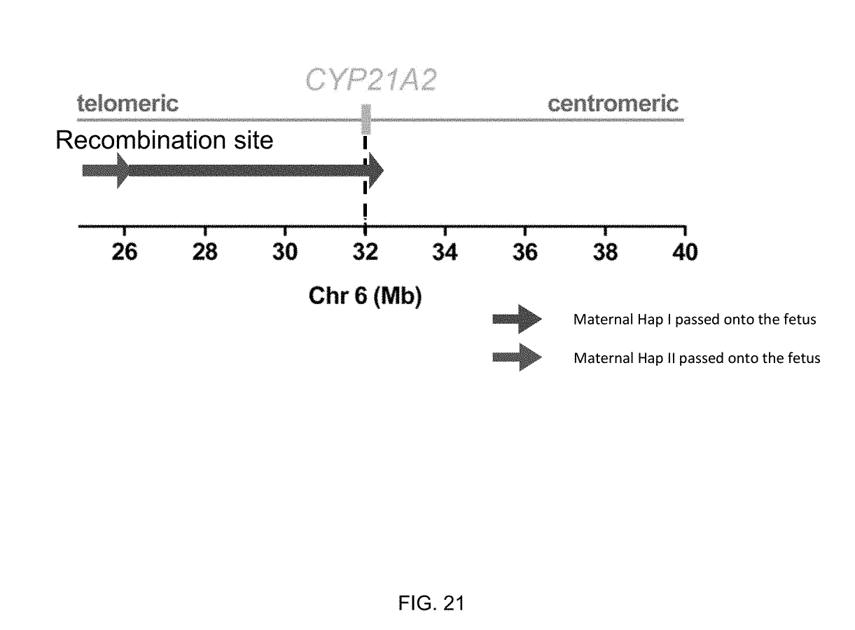

FIG. 21 shows a recombination identification with the use sliding window based PRHDO according to embodiments of the present invention. Using such accumulatively dynamic approaches, we could correctly identify the recombination site present in case MP21. Accordingly, embodiments can repeat for other chromosomal regions that form sliding windows that overlap with each other. A recombination can be identified when a specified number of consecutive sliding windows indicate a change to a new maternal haplotype being inherited.

5. RHSO Accuracy in Error-Prone Regions

In some situation, a size-based approach can give a better performance than count-based analysis. For example, for error-prone regions including repetitive, low-complexity, high-GC% regions, mapping and hybridization would introduce extra biases which would affect the count representation, thus affecting the accuracy and sensitivity of RHDO. However, for size analysis, the focus on the size profile within the regions of interest can minimize the influence derived from different regions sharing some sequence similarities.

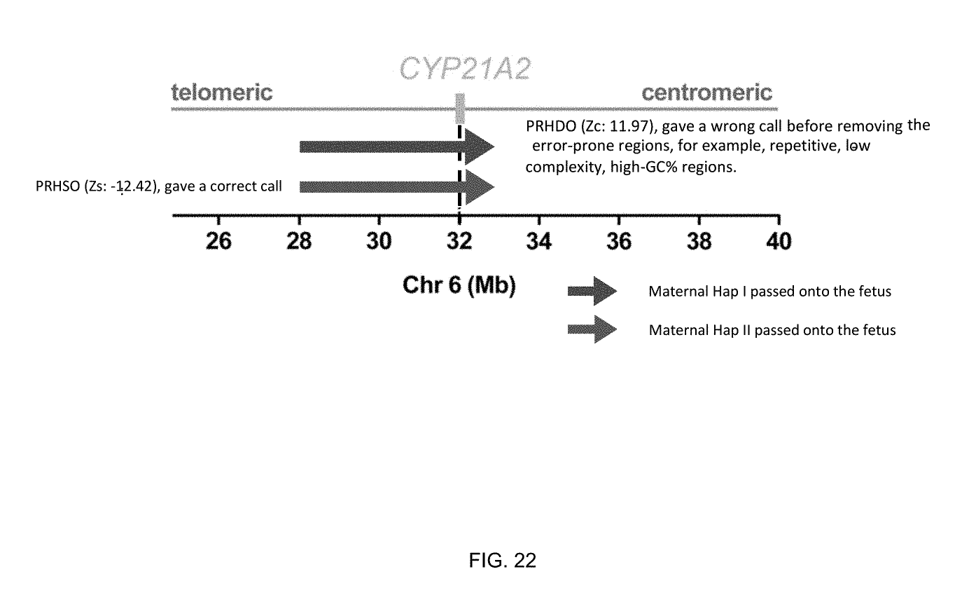

To illustrate this point, we reanalyzed the case MP10623 using those DNA fragments located on the aforementioned error-prone regions. As a result, PRHDO would give rise to a call with a Zc value of 11.97, suggesting Hap I passed onto the fetuses but the fetus in fact inherited the maternal Hap II according to the actual clinical information. In contrast, PRHSO still gave a correct call with a Zs value of −12.42 (FIG. 22). Such property of PRHSO would be particularly important to analyze those genes located within telomere and centromere regions for example but not limited to F8 and HBA1 genes.

FIG. 22 shows results for PRHSO and PRHDO for an error-prone region according to embodiments of the present invention. In FIG. 22, suggests that RHSO would be more robust in those error-prone regions, which would be superior to PRHDO when the disease-associated genes located within telomere and centromere regions, for example but not limited to F8 gene, which is related to hemophilia and HBA1 that is related to thalassemias.

D. Discussion

PRHSO and PRHDO approaches further extend the application to determine the maternal inheritance without paternal haplotype information. Obviating the need to sequence paternal samples, the cost of the assay can be reduced. On the other hand, it is not uncommon that the paternal specimen is not available in real clinical setting. PRHSO and PRHDO can still enable the examination on whether the disease-associated maternal haplotype was passed on to the fetus. Therefore, prenatal detection of maternally inherited autosomal dominant disorders (Saito H et al., Lancet 2000; 356:1170) or exclusion of autosomal recessive disorders (Chiu R W et al., Lancet 2002; 360:998-1000) can be achieved.

Fetal DNA fraction and the size profile of the maternally- and fetally-derived DNA in maternal plasma are two key variables influencing the number of DNA molecules required in RHSO analysis. For example, the fetal DNA fraction was only 1.4% in case MP3 and thus more sequenced reads were needed for classification. For case MP4, the exceptionally high number of DNA molecules required in RHSO could be explained by, virtually, the absence of difference between the maternal and fetal plasma DNA size profiles.

In addition, since RHSO approach included size filtering, more DNA molecules would be needed to achieve the same sensitivity in RHSO than PRHDO analysis. Therefore, HAI and M12418 could be classified in only PRHDO analysis because of the inadequate number of plasma DNA molecules available for RHSO analysis.

The sequencing depth and the number of SNPs used in the analysis are two major factors that affect the accuracy of RHSO. In general, the more the heterozygous SNP loci are analyzed, the lower the sequencing depth is required to achieve the same level of accuracy. In our simulation analysis, RHSO could accurately deduce the fetal inheritance of maternally transmitted mutations inheritance of the fetus, provided that the fetal DNA fraction is 3% and 900 heterozygous SNPs, with the sequencing depth of 100 fold (FIG. 18A). In this study, the number of plasma DNA molecules used in the unclassified cases was lower than that of the theoretical number of molecules required. In some implementations, the classification could be achieved by analyzing more DNA molecules through increasing the sequencing depth or expanding capture probes to target more SNPs.

Our empirical data and the simulation data show that PRHDO requires less amount of plasma DNA analyzed, or less sequencing than PRHSO, thus PRHDO could be performed by single-end sequencing. However, if an adequate number of informative DNA molecules were analyzed for both PRHDO and PRHSO, they could provide confirmation of the classification result to each other. Similarly, RHDO and RHSO can provide confirmation to each other. Thus, PRHSO could be adopted as a complementary or synergistic method to PRHDO for non-invasively detection of maternal haplotype inheritance, including single-gene diseases using maternal plasma DNA. For example, PRHSO can provide additional value in NIPT detection of single-gene diseases when expanding the gene panel (i.e., number of mutations targeted) for population screening When the gene panel is expanded, using just one technique can cause the false positive rate to increase, but the use of both techniques can reduce the false positive rate. With some high risk mutations, it may be desirable to identify a mutation as being inherited when either technique indicates inheritance, so as to improve sensitivity.

E. Method of Determining Inherited Haplotype without Partner Information

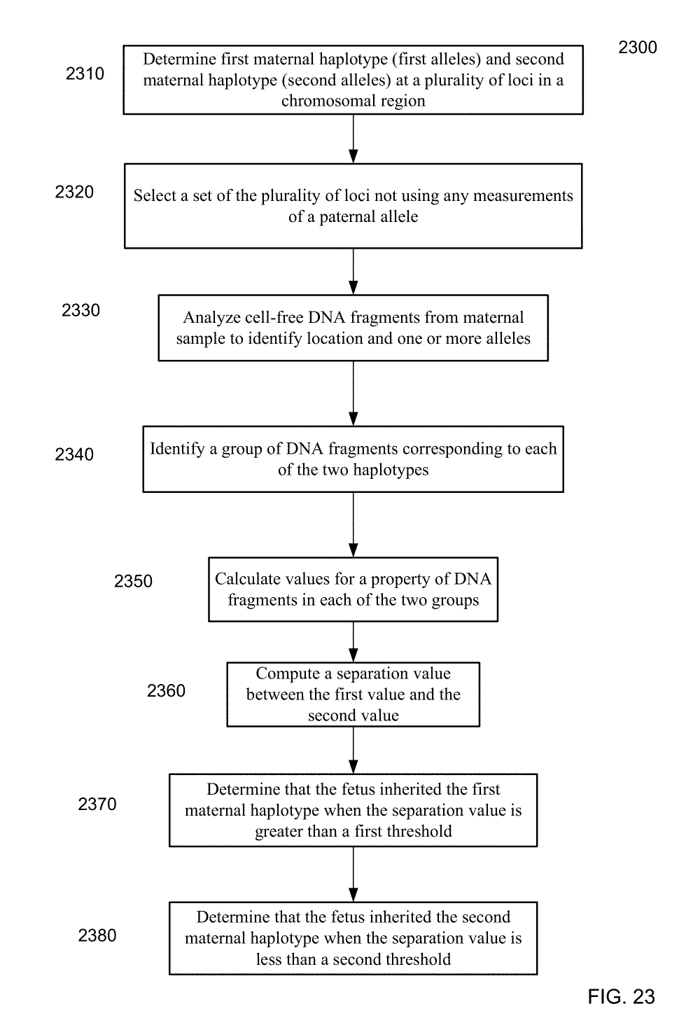

FIG. 23 is a flowchart of a method 2300 of determining a portion of a fetal genome of a fetus inherited from a pregnant mother using a biological sample obtained from the pregnant mother. The pregnant mother has a maternal genome with a first maternal haplotype and a second maternal haplotype in a chromosomal region. The biological sample comprises a mixture of maternal and fetal DNA fragments.

At block 2310, a first maternal haplotype and a second maternal haplotype are determined. The determination be made based on an analysis of DNA in one or more other samples. For example, the biological sample can be a maternal plasma sample from a blood sample, and the other sample can be the buffy coat from the blood sample. Thus, the maternal plasma sample is different than the buffy coat. The sequencing can include linked-read sequencing of DNA molecules, e.g., which are at least 1 kb long.

The first maternal haplotype can be determined to have first alleles at a plurality of loci in the chromosomal region, where the maternal genome is heterozygous at the plurality of loci. The second maternal haplotype can be determined to have second alleles at the plurality of loci in the chromosomal region, where the second alleles are different than the first alleles. Block 2310 may be performed in a similar manner as block 410 of FIG. 4.

At block 2320, a set of the plurality of loci is selected. The selection of the set of loci may not use any measurements of a paternal allele. For example, the heterozygous loci in the region may just be selected, even though the inherited paternal haplotype is unknown. In some embodiments, population statistics about the percentage of people (e.g., in a subpopulation that includes the mother) may be used to select loci where the fetus is likely to be homozygous. Accordingly, the selection of the set of loci can access a database of population statistics for a population that corresponds to the father of the fetus and/or the fetus itself (e.g., if population is different for fetus due to the mother being from a different population than the father), where a locus having a prevalence of being heterozygous that is above a cutoff value for the population is excluded. The prevalence of being heterozygous can be considered equivalent to a prevalence of being homozygous, as the two are related.

The fetal genome may be homozygous at some of the set of loci (e.g., a first portion) and heterozygous at some of the set of loci (e.g., a second portion) as a result of not knowing the paternally-inherited allele. The location where the fetus is heterozygous generally do not indicate which haplotype is inherited, but since the fetus is homozygous at some of the loci, an imbalance in the two haplotypes can be detected. The proportion of loci at which the fetus is homozygous can vary, e.g., from 20%, 30, 40%, 50%, 60%, 70%, 80%, 90%, or 100%.

In some embodiments, the set of the plurality of loci can include identifying a mutation at a first location in the first maternal haplotype in the chromosomal region and selecting the set of loci that are within a specified distance of the first location of the mutation. Example distances are provided herein.

At block 2330, a plurality of cell-free DNA fragments are analyzed from the biological sample obtained from the pregnant mother. Block 2330 may be performed in a similar manner as block 320 of FIG. 3. The plurality of cell-free DNA fragments can be analyzed via a targeted procedure, e.g., when a mutation is being detected. For example, a sequencing of the plurality of cell-free DNA fragments can target a genomic window that includes the mutation. Another embodiment can use probes and/or primers that are specific to a genomic window that includes the mutation.

At block 2340, groups of DNA fragments corresponding to each of the haplotypes are identified. Block 2340 may be performed in a similar manner as block 330 of FIG. 3 and block 430 of FIG. 4. For example, a first group of DNA fragments can be identified as corresponding to the first maternal haplotype based on each of these DNA fragments having one of the first alleles. A second group of DNA fragments can be identified as corresponding to the second maternal haplotype based on each of these DNA fragments having one of the second alleles.

At block 2350, a property of DNA fragments in each of the two groups is calculated. Block 2350 may be performed in a similar manner as block 435 of FIG. 4. In various embodiments, the properties can be determined according to RHDO or RHSO. For example, the first value can be an average size of the DNA fragments of the first group, and the second value can be an average size of the DNA fragments of the second group. As another example, the first value Q_{HapI} is a fraction of DNA fragments in the first group that are shorter than a cutoff size, and the second value Q_{HapII} is a fraction of DNA fragments in the second group that are shorter than the cutoff size. As another example, the first value F_{Hap I} and the second value F_{Hap II} are defined for a respective haplotype as F=Σ^{w}length/Σ^{N}length, where Σ^{w}length represents a sum of lengths of the DNA fragments of a corresponding group with a length equal to or less than a cutoff size w; and μ^{N}length represents a sum of lengths of the DNA fragments of the corresponding group with a length equal to or less than N bases, where N is greater than w.

At block 2360, a separation value is computed between the first value and the second value. Block 2360 may be performed in a similar manner as block 3340 of FIG. 3.

At block 2370, it is determined that the fetus inherited the first maternal haplotype when the separation value is greater than a first threshold. In various embodiments, the first threshold can be an absolute number, a percentage, or other normalized value (e.g., modulated by a variance). For example, when the separation value is Zs or Zc (as in equations (1) and (3)), the first threshold could be 3. A different threshold can be selected depending on desired specificity and sensitivity, as well as based on a variety of other factors, e.g., population statistics for the set of loci chosen and a measured fetal concentration. As another example, the separation value could include a ratio, which affects the numerator in the z-score to be determined (e.g., ΔF being a ratio), but the usage of the variance can still be a threshold of 3 (or other number of standard deviations) to be used.

In some embodiments, the first threshold and second threshold are selected using a statistical distribution for defining a stochastic variation that estimates a standard deviation. For example, the statistical value can be divided by the expected amount of variation for the given statistical distribution (e.g., the Poisson distribution, as described herein).

At block 2380, it is determined that the fetus inherited the second maternal haplotype when the separation value is less than a second threshold. For example, when the separation value is Zs or Zc (as in equations (1) and (3)), the first threshold could be −3, or other negative value, at least when a z-score is used. In some embodiments, both thresholds could be positive, e.g., when a ratio is taken between the two values. For example, one threshold could be 2 for the haplotype corresponding to the numerator in the ratio, and the other threshold could be ½ for the haplotype that is in the denominator.

Other types of ratios could be used as well. For example, the denominator could include a sum of counts for both haplotypes. Such a change would affect the thresholds used, but such thresholds would have a defined relationship between the different techniques for determining the separation values. In such an example with the sum of values in the denominator, two separation values can be determined, and each separation value could be compared to a same threshold, thereby confirming which haplotype is overrepresented. Such a technique is the same as determining one separation value and comparing to two thresholds, as it is simply applying a transformation to the separation value and to the second threshold.

### III. Example Systems

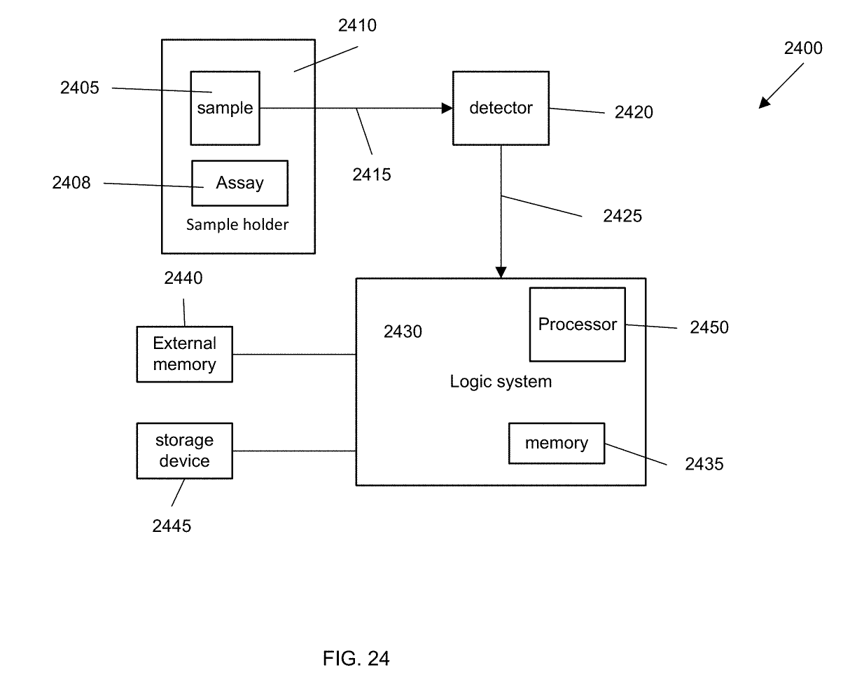

FIG. 24 illustrates a measurement system 2400 according to an embodiment of the present invention. The system as shown includes a sample 2405, such as cell-free DNA molecules within a sample holder 2410, where sample 2405 can be contacted with an assay 2408 to provide a signal of a physical characteristic 2415. An example of a sample holder can be a flow cell that includes probes and/or primers of an assay or a tube through which a droplet moves (with the droplet including the assay). Physical characteristic 2415 (e.g., a fluorescence intensity, a voltage, or a current), from the sample is detected by detector 2420. Detector can take a measurement at intervals (e.g., periodic intervals) to obtain data points that make up a data signal. In one embodiment, an analog to digital converter converts an analog signal from the detector into digital form at a plurality of times. A data signal 2425 is sent from detector 2420 to logic system 2430. Data signal 2425 may be stored in a local memory 2435, an external memory 2440, or a storage device 2445.

Logic system 2430 may be, or may include, a computer system, ASIC, microprocessor, etc. It may also include or be coupled with a display (e.g., monitor, LED display, etc.) and a user input device (e.g., mouse, keyboard, buttons, etc.). Logic system 2430 and the other components may be part of a stand-alone or network connected computer system, or they may be directly attached to or incorporated in a device (e.g., a sequencing device) that includes detector 2420 and/or sample holder 2410. Logic system 2430 may also include software that executes in a processor 2450. Logic system 2430 may include a computer readable medium storing instructions for controlling measurement system 2400 to perform any of the methods described herein.

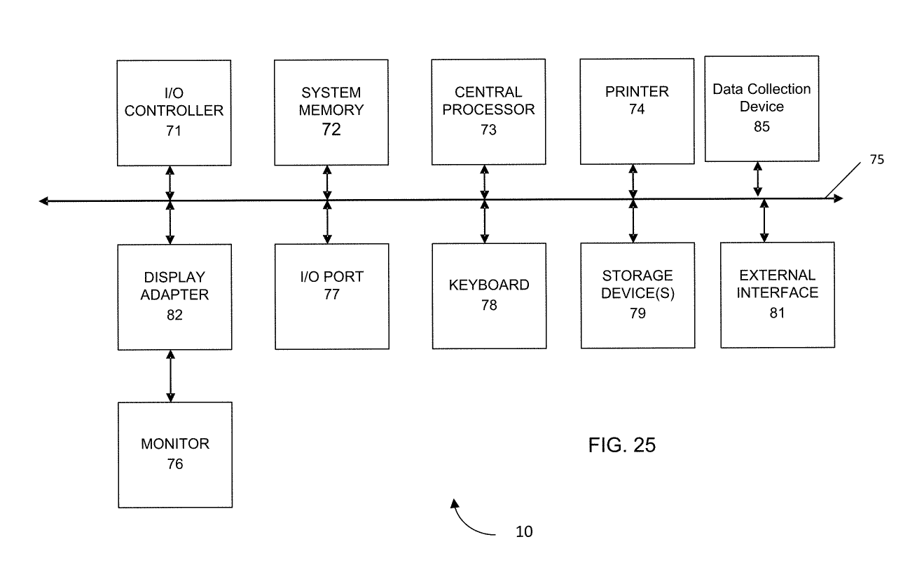

Any of the computer systems mentioned herein may utilize any suitable number of subsystems. Examples of such subsystems are shown in FIG. 25 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 25 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art.

What is claimed is:

1. A method of determining a portion of a fetal genome of a fetus inherited from a pregnant mother using a biological sample obtained from the pregnant mother, the pregnant mother having a maternal genome with a first maternal haplotype and a second maternal haplotype in a chromosomal region, where the biological sample comprises a mixture of maternal and fetal DNA fragments, the method comprising:
based on an analysis of DNA in one or more other samples:
determining the first maternal haplotype to have first alleles at a plurality of loci in the chromosomal region, the maternal genome being heterozygous at the plurality of loci, and
determining the second maternal haplotype to have second alleles at the plurality of loci in the chromosomal region, the second alleles being different than the first alleles;
selecting a set of the plurality of loci, wherein selecting the set of loci does not use any measurements of a paternal allele;
analyzing a plurality of cell-free DNA fragments from the biological sample obtained from the pregnant mother, wherein analyzing a DNA fragment includes:
identifying a location of the DNA fragment in a reference genome; and
determining an allele of the DNA fragment;
identifying a first group of DNA fragments in the biological sample as having one of the first alleles at one of the set of loci based on the identified locations and the determined alleles for the first group of DNA fragments;
identifying a second group of DNA fragments in the biological sample as having one of the second alleles at one of the set of loci based on the identified locations and the determined alleles for the second group of DNA fragments;
calculating, by a computer system, a first value of the first group of DNA fragments, the first value defining a property of the DNA fragments of the first group;
calculating, by the computer system, a second value of the second group of DNA fragments, the second value defining a property of the DNA fragments of the second group;
computing a separation value between the first value and the second value;
determining that the fetus inherited the first maternal haplotype when the separation value is greater than a first threshold; and
determining that the fetus inherited the second maternal haplotype when the separation value is less than a second threshold.

2. The method of claim 1, further comprising:
repeating the method for other chromosomal regions that form sliding windows that overlap with each other; and
identifying a recombination when a specified number of consecutive sliding windows indicate a change to a new maternal haplotype being inherited.

3. The method of claim 1, wherein the fetal genome is homozygous at some of the set of loci and heterozygous at some of the set of loci.

4. The method of claim 1, wherein the fetal genome is homozygous at 30% or more of the set of loci.

5. The method of claim 1, wherein the first threshold and second threshold are selected using a statistical distribution for defining a stochastic variation that estimates a standard deviation.

6. The method of claim 1, wherein the separation value includes a difference between the first value and the second value, and wherein one of the first and second threshold is positive and another of the first and second thresholds is negative.

7. The method of claim 1, wherein the separation value includes a ratio between the first value and the second value.

8. The method of claim 1, wherein the first value corresponds to a statistical value of a size distribution of the DNA fragments of the first group, and the second value corresponds to a statistical value of a size distribution of the DNA fragments of the second group.

9. The method of claim 8, wherein the first value is an average size of the DNA fragments of the first group, and the second value is an average size of the DNA fragments of the second group.

10. The method of claim 8, wherein the first value Q_{HapI} is a fraction of DNA fragments in the first group that are shorter than a cutoff size, and the second value Q_{HapII} is a fraction of DNA fragments in the second group that are shorter than the cutoff size.

11. The method of claim 10, wherein the separation value includes a difference between the first value and the second value, and wherein the difference includes ΔQ=Q_{HapI}−Q_{HapII}.

12. The method of claim 8, wherein the first value F_{Hap I} and the second value F_{Hap II} a are defined for a respective haplotype as F=Σ^{w}length/Σ^{N}length, where Σ^{w}length represents a sum of lengths of the DNA fragments of a corresponding group with a length equal to or less than a cutoff size w; and Σ^{N}length represents a sum of lengths of the DNA fragments of the corresponding group with a length equal to or less than N bases, where N is greater than w.

13. The method of claim 12, wherein the separation value includes a difference between the first value and the second value, wherein the difference includes ΔF=F_{HapI}−F_{Hap II}.

14. The method of claim 1, wherein the first value of the first group corresponds to a number of DNA fragments located at the set of loci, and the second value of the second group corresponds to a number of DNA fragments located at the set of loci.

15. The method of claim 1, further comprising:
determining a fractional concentration of fetal DNA in the biological sample; and
using the fractional concentration to determine the first threshold and the second threshold.

16. The method of claim 1, wherein the one or more other samples are of cellular tissue from the pregnant mother, the method further comprising:
sequencing DNA molecules that overlap with the chromosomal region and that are at least 1 kb long in the one or more other samples to determine the first maternal haplotype and the second maternal haplotype.

17. The method of claim 16, wherein the sequencing includes single molecule sequencing.

18. The method of claim 16, wherein the sequencing includes linked-read sequencing of DNA molecules that are at least 1 kb long.

19. The method of claim 1, wherein selecting the set of the plurality of loci includes:
identifying a mutation at a first location in the first maternal haplotype in the chromosomal region; and
selecting the set of loci that are within a specified distance of the first location of the mutation.

20. The method of claim 19, wherein the specified distance is 5 Mb.

21. The method of claim 19, wherein analyzing the plurality of cell-free DNA fragments from the biological sample includes:
sequencing the plurality of cell-free DNA fragments, wherein the sequencing targets a genomic window that includes the mutation.

22. The method of claim 19, wherein analyzing the plurality of cell-free DNA fragments from the biological sample includes:
using probes and/or primers that are specific to a genomic window that includes the mutation.

23. The method of claim 1, wherein selecting the set of the plurality of loci includes:
accessing a database of population statistics for a population that corresponds to the fetus and/or to a father of the fetus; and
excluding a locus having a prevalence of being heterozygous that is above a cutoff value for the population.

24. The method of claim 1, wherein biological is plasma from a blood sample, and wherein the one or more other samples includes a buffy coat from the blood sample.

25. The method of claim 1, wherein the first group includes at least one DNA fragment located at each of the set of loci, and wherein the second group includes at least one DNA fragment located at each of the set of loci.

26. A method for detecting a mutation in a fetal genome in a fetal genome of a fetus inherited from a pregnant mother using a biological sample obtained from the pregnant mother, the pregnant mother having a maternal genome with a first maternal haplotype and a second maternal haplotype in a chromosomal region, wherein the biological sample contains a mixture of maternal and fetal DNA fragments, the method comprising:
sequencing DNA molecules that overlap with the chromosomal region and that are at least 1 kb long in a cellular maternal sample to obtain long sequence reads from both chromosomal copies in the chromosomal region;
constructing the first maternal haplotype using a first set of long sequence reads that share alleles at a plurality of loci in the chromosomal region, the first maternal haplotype having first alleles at the plurality of loci;
constructing the second maternal haplotype using a second set of long sequence reads that share alleles at the plurality of loci in the chromosomal region, the second maternal haplotype having second alleles at the plurality of loci;
identifying a mutation at a first location in the first maternal haplotype in the chromosomal region;
analyzing a plurality of cell-free DNA fragments from the biological sample obtained from the pregnant mother, wherein analyzing a DNA fragment includes:
identifying a location of the DNA fragment in a reference genome; and
determining an allele of the DNA fragment;
selecting a set of the plurality of loci based on the first location of the mutation;
determining paternal alleles inherited by the fetus from a father at the set of loci, wherein the paternal alleles correspond to the first alleles or the second alleles, and wherein the set of loci is further selected based on locations that the paternal alleles are determined;
identifying a first group of DNA fragments in the biological sample as having one of the first alleles at one of the set of loci based on the identified locations and the determined alleles for the first group of DNA fragments;
identifying a second group of DNA fragments in the biological sample as having one of the second alleles at one of the set of loci based on the identified locations and the determined alleles for the second group of DNA fragments;
calculating, by a computer system, a first value of the first group of DNA fragments, the first value defining a property of the DNA fragments of the first group;
calculating, by the computer system, a second value of the second group of DNA fragments, the second value defining a property of the DNA fragments of the second group;
computing a separation value between the first value and the second value; and
determining whether the fetus inherited the mutation on the first maternal haplotype based on a comparison of the separation value to a cutoff value and based on whether the paternal alleles correspond to the first alleles or the second alleles.

27. The method of claim 26, wherein the sequencing includes linked-read sequencing of DNA molecules to reconstruct the long sequence reads from smaller linked reads, wherein the chromosomal region includes a structural variation, and wherein constructing the first maternal haplotype includes identifying reconstructed long sequence reads that each differ in length from an average length of reconstructed long sequence reads for regions before and after the structural variation by at least a specified length.

28. The method of claim 26, wherein analyzing the plurality of cell-free DNA fragments from the biological sample includes:
sequencing the plurality of cell-free DNA fragments, wherein the sequencing targets a genomic window that includes the mutation.

29. The method of claim 26, wherein analyzing the plurality of cell-free DNA fragments from the biological sample includes:
using probes and/or primers that are specific to a genomic window that includes the mutation.

30. The method of claim 26, wherein the first group includes at least one DNA fragment located at each of the set of loci, and wherein the second group includes at least one DNA fragment located at each of the set of loci.

31. The method of claim 26, wherein the set of loci are selected to be within a specified distance of the first location of the mutation.

32. The method of claim 31, wherein the specified distance is 5 Mb.

33. A method for detecting a mutation in a fetal genome of a fetus inherited from a father using a biological sample obtained from a pregnant mother of the fetus, the father having a paternal genome with a first paternal haplotype and a second paternal haplotype in a chromosomal region, wherein the biological sample contains a mixture of maternal and fetal DNA fragments, the method comprising:
sequencing DNA molecules that overlap with the chromosomal region and that are at least 1 kb long in a cellular paternal sample to obtain long sequence reads from both chromosomal copies in the chromosomal region, the long sequence reads being at least 1 kb long;
constructing the first paternal haplotype using a first set of long sequence reads that share alleles at a plurality of loci in the chromosomal region, the first paternal haplotype having first alleles at the plurality of loci;
constructing the second paternal haplotype using a second set of long sequence reads that share alleles at the plurality of loci in the chromosomal region, the second paternal haplotype having first alleles at the plurality of loci;
identifying the mutation at a first location in the first paternal haplotype in the chromosomal region;
analyzing a plurality of cell-free DNA fragments from the biological sample obtained from the pregnant mother, wherein analyzing a DNA fragment includes:
identifying a location of the DNA fragment in a reference genome; and
determining an allele of the DNA fragment;
selecting a set of the plurality of loci based on the first location of the mutation and based on a maternal genome of the pregnant mother being homozygous at the set of loci, wherein the maternal genome is homozygous for the first alleles at a first subset of the set of loci, and wherein the maternal genome is homozygous for the second alleles at a second subset of the set of loci;
identifying a first group of DNA fragments in the biological sample as having one of the first alleles at one of the first subset of loci based on the identified locations and the determined alleles for the first group of DNA fragments;
identifying a second group of DNA fragments in the biological sample as having one of the second alleles at one of the second subset of loci based on the identified locations and the determined alleles for the second group of DNA fragments;
calculating, by a computer system, a first amount of the first group of DNA fragments;
calculating, by the computer system, a second amount of the second group of DNA fragments;
computing a separation value between the first amount and the second amount; and
determining whether the fetus inherited the mutation on the first paternal haplotype based on a comparison of the separation value to a cutoff value.

34. The method of claim 33, wherein the sequencing includes linked-read sequencing of DNA molecules to reconstruct the long sequence reads from smaller linked reads, wherein the chromosomal region includes a structural variation, and wherein constructing the first maternal haplotype includes identifying reconstructed long sequence reads that each differ in length from an average length of reconstructed long sequence reads for regions before and after the structural variation by at least a specified length.

35. The method of claim 33, wherein analyzing the plurality of cell-free DNA fragments from the biological sample includes:
sequencing the plurality of cell-free DNA fragments, wherein the sequencing targets a genomic window that includes the mutation.

36. The method of claim 33, wherein analyzing the plurality of cell-free DNA fragments from the biological sample includes:
using probes and/or primers that are specific to a genomic window that includes the mutation.

37. The method of claim 33, wherein the first group includes at least one DNA fragment located at each of the first subset of loci, and wherein the second group including at least one DNA fragment located at each of the second subset of loci.

38. The method of claim 33, wherein the set of loci are selected to be within a specified distance of the first location of the mutation.

39. The method of claim 38, wherein the specified distance is 5 Mb.
